(19) United States
(12) Patent Application Publication
(10) Pub. No.: US 2025/0034529 A1
(43) Pub. Date: Jan. 30, 2025

(54) Methods and Uses of Differentiated Cells

(71) Applicant: Miromatrix Medical Inc., Eden Prairie, MN (US)

(72) Inventors: David LO, Hopkins, MN (US); MacKenna STACHEL, Apple Valley, MN (US); Emily BECK, Hopkins, MN (US); Ethan VANDERSLICE, Minneapolis, MN (US); Jody BONNEVIER, Hopkins, MN (US)

(21) Appl. No.: 18/680,248

(22) Filed: May 31, 2024

Related U.S. Application Data
(60) Provisional application No. 63/470,248, filed on Jun. 1, 2023.

(51) Int. Cl. C12N 5/071

(52) U.S. Cl. CPC C12N 5/0686; C12N 2501/105; C12N 2501/11; C12N 2501/115; C12N 2501/165; C12N 2533/90

(57) ABSTRACT

Disclosed herein are methods for making, culturing, and maintaining engineered podocyte-like cells. The methods comprise growing glomerular cells in in vitro conditions to differentiate the glomerular cells into podocyte-like cells. Also disclosed herein are isolated and purified podocyte-like cells and kits comprising the engineered podocyte-like cells and/or the media used to differentiate the podocyte-like cells. The engineered podocyte-like cells can be used in recellularization of a decellularized extracellular matrix. In some instances, disclosed herein are recellularized isolated organs that can comprise the engineered podocyte-like cells.

### CROSS REFERENCE TO RELATED APPLICATIONS

This application claims the benefit of, and priority to, U.S. Provisional Patent Application Ser. No. 63/470,248, filed Jun. 1, 2023, the entirety of which is incorporated by reference and commonly owned.

### SUMMARY

Disclosed herein are methods of making engineered podocyte-like cells. In some embodiments the method can comprise: a) culturing glomerular cells in a first media comprising at least one of a retinoic acid, a corticosteroid, a calcitriol, or a salt of any one of these for about 2-4 days; b) removing the glomerular cells from the first media; and c) culturing the glomerular cells in a second media comprising at least one of a SB431542, a salt thereof, an IWR-1-endo, or a salt thereof for about 6-12 days, wherein the culturing of the glomerular cells in the second media for about 6-12 days results in differentiation of the glomerular cells into the engineered podocyte-like cells.

In some embodiments after the glomerular cells are cultured in the second media the glomerular cells can be differentiated into the engineered podocyte-like cells, which have increased expression of one or more of: podocin, nephrin, podocalyxin, or synaptopodin as compared to the glomerular cells prior to growth in the first media. In some embodiments the corticosteroid can comprise a dexamethasone or a salt thereof. In some embodiments the first media further can comprise a basal medium, a nutrient mix, an antibiotic, an insulin-transferrin-selenium (ITS), a fetal bovine serum, a salt of any of these, or any combination thereof. In some embodiments, the antibiotic can comprise a penicillin, a salt thereof, a streptomycin, a salt thereof, or any combination thereof. In some embodiments the concentration of the retinoic acid, or the salt thereof in the first media can be from about 1 μM to about 1 mM. In some embodiments the concentration of the corticosteroid, or the salt thereof in the first media can be from about 100 nM to about 10 mM. In some embodiments the concentration of the calcitriol or the salt thereof in the first media can be from about 1 nM to about 300 nM. In some embodiments the second media can further comprise a basal medium, a nutrient mix, an antibiotic, an insulin-transferrin-selenium (ITS), a fetal bovine serum, a salt of any of these, or any combination thereof. In some embodiments, the antibiotic can comprise a penicillin, a salt thereof, a streptomycin, a salt thereof, or any combination thereof. In some embodiments, the concentration of the SB431542, or the salt thereof in the second media can be from about 1 μM to about 10 μM. In some embodiments, the concentration of the IWR-1-endo, or the salt thereof in the second media can be from about 1 μM to about 10 μM. In some embodiments, the engineered podocyte-like cells can have increased gene expression of: NPHS1, NPHS2, SYNPO, or any combination thereof as compared to the glomerular cells. In some embodiments, the increased gene expression can be determined by quantitative reverse-transcriptase PCR. In some embodiments, the glomerular cells can be glomerular outgrowth cells. In some embodiments, the engineered podocyte-like cells can comprise a cytoskeletal organization with multiple extensions as compared to the cytoskeletal organization of glomerular cells. In some embodiments, growing the glomerular cells in the first media for about 3 days further can comprise replacing the first media with a fresh first media after about 48 hours of cell growth. In some embodiments, growing the glomerular cells in the second media for about 7 days can comprise replacing the second media with a fresh second media every 48 hours of cell growth. In some embodiments, the engineered podocyte-like cells can have decreased expression of one or more of: podocin, nephrin, podocalyxin, or synaptopodin as compared to primary podocytes. In some embodiments, the glomerular cells can be animal glomerular cells. In some embodiments, the animal glomerular cells can be human glomerular cells. In some embodiments, the animal glomerular cells can be pig glomerular cells, sheep glomerular cells, goat glomerular cells, monkey glomerular cells, cow glomerular cells, dog glomerular cells, or cat glomerular cells. In some embodiments, the first media can comprise the retinoic acid, or the salt thereof. In some embodiments, the first media can comprise the corticosteroid, or the salt thereof. In some embodiments, the first media can comprise the calcitriol, or the salt thereof. In some embodiments, the second media can comprise the SB431542, or the salt thereof. In some embodiments, the second media can comprise the IWR-1-endo, or the salt thereof. In some embodiments, the glomerular cells can be grown in the first media for about 3 days. In some embodiments, the glomerular cells can be grown in the second media for about 7 days. In some embodiments, the glomerular cells can be grown in an least partially decellularized kidney extracellular matrix. In some embodiments, the increased expression of one or more of: podocin, nephrin, podocalyxin, or synaptopodin can be determined by a fluorescence microscopy, a Western blot, a flow cytometry, or any combination thereof. Also disclosed herein are engineered podocyte-like cell made by the methods described above

Also disclosed herein are engineered podocyte-like cells. In some embodiments, the engineered podocyte-like cell can comprise: a) increased expression of one or more of: podocin, nephrin, podocalyxin or synaptopodin as compared to a glomerular cell; and b) decreased expression of one or more of: podocin, nephrin, podocalyxin or synaptopodin as compared to a primary podocyte cell. In some embodiments, the engineered podocyte-like cells can comprise a cytoskeletal organization with multiple extensions as compared to the cytoskeletal organization of the glomerular cell. In some embodiments, the engineered podocyte-like cells can have increased gene expression of: NPHS1, NPHS2, SYNPO, or any combination thereof as compared to the glomerular cell. In some embodiments, the engineered podocyte-like cells can have decreased localization of one or more of: podocin, nephrin, podocalyxin, or synaptopodin as compared to primary podocytes.

Also disclosed herein are methods of engrafting cells on an at least partially decellularized kidney extracellular matrix comprising: contacting the at least partially decellularized kidney extracellular matrix with a plurality of the engineered podocyte-like cells. In some embodiments, the engineered podocyte-like cell can comprise: a) increased expression of one or more of: podocin, nephrin, podocalyxin or synaptopodin as compared to a glomerular cell; and b) decreased expression of one or more of: podocin, nephrin, podocalyxin or synaptopodin as compared to a primary podocyte cell. In some embodiments, the engineered podocyte-like cells can comprise a cytoskeletal organization with multiple extensions as compared to the cytoskeletal organization of the glomerular cell. In some embodiments, the engineered podocyte-like cells can have increased gene expression of: NPHS1, NPHS2, SYNPO, or any combination thereof as compared to the glomerular cell. In some embodiments, the engineered podocyte-like cells can have decreased localization of one or more of: podocin, nephrin, podocalyxin, or synaptopodin as compared to primary podocytes. In some embodiments, the contacting can occur in a bioreactor chamber. In some embodiments, the contacting comprises depositing through a ureter of the at least partially decellularized kidney extracellular matrix the plurality of the engineered podocyte-like cells in an aqueous composition into a glomerulus of the at least partially decellularized kidney extracellular matrix, thereby engrafting cells on the at least partially decellularized kidney extracellular matrix. In some embodiments, the method can further comprise seeding a plurality of mesangial cells, a plurality of human umbilical vein endothelial cells (HUVEC), or both. In some embodiments, the depositing through the ureter can comprise creating a vacuum in the bioreactor chamber. In some embodiments, the method can further comprise continuously perfusing a media through the at least partially decellularized kidney extracellular matrix after the engrafting. In some embodiments, the media can be changed about every 24 hours.

Also disclosed herein are at least partially recellularized isolated organs or portions thereof comprising the engineered podocyte-like cell. In some embodiments, the at least partially recellularized isolated organ or portion thereof in a closed loop normothermic perfusion system a) can sustain urine/serum protein values in urine of less than 30% at 1 hour post normothermic perfusion, and less than 65% at 4 hours post implantation; or b) can sustain urine/serum hematocrit levels in urine of less than 30% at 1 hour post normothermic perfusion, and less than 1% at 4 hours post implantation. In some embodiments, the engineered podocyte-like cell can comprise: a) increased expression of one or more of: podocin, nephrin, podocalyxin or synaptopodin as compared to a glomerular cell; and b) decreased expression of one or more of: podocin, nephrin, podocalyxin or synaptopodin as compared to a primary podocyte cell. In some embodiments, the engineered podocyte-like cells can comprise a cytoskeletal organization with multiple extensions as compared to the cytoskeletal organization of the glomerular cell. In some embodiments, the engineered podocyte-like cells can have increased gene expression of: NPHS1, NPHS2, SYNPO, or any combination thereof as compared to the glomerular cell. In some embodiments, the engineered podocyte-like cells can have decreased localization of one or more of: podocin, nephrin, podocalyxin, or synaptopodin as compared to primary podocytes. In some embodiments, the at least partially recellularized isolated organ or portion thereof can comprise a kidney or a portion thereof. In some embodiments, in the closed loop normothermic perfusion system levels of creatinine, urea, sodium, potassium, glucose, lactate, bicarbonate, or any combination thereof can be determined. In some embodiments, at least partially recellularized isolated organ or portion thereof can be allogeneic to the engineered podocyte-like cell. In some embodiments, at least partially recellularized isolated organ or portion thereof can be autologous to the engineered podocyte-like cell. In some embodiments, at least partially recellularized isolated organ or portion thereof can be xenogeneic to the engineered podocyte-like cell.

Also disclosed herein are kits comprising a first media for growing podocyte-like cells comprising at least one of: a retinoic acid, a salt thereof, a corticosteroid, a salt thereof, a calcitriol, or a salt thereof in a container, and a second media for growing podocyte-like cells comprising at least one of: a SB431542, a salt thereof, an IWR-1-endo, or a salt thereof in a container. In some embodiments, the first media, the second media, or both can comprise a glomerular cell.

Also disclosed is a method of making engineered podocyte-like cells, the method comprising: a) culturing glomerular cells in a first media comprising at least one of a transforming growth factor beta pathway inhibitor, and a Wnt pathway inhibitor, or a salt of either of these for about 4-8 days; and b) removing the glomerular cells from the first media; and c) culturing the glomerular cells in a second media comprising at least one of a retinoic acid, a Rho kinase (ROCK) inhibitor, or a salt of either of these for about 2-6 days, wherein the culturing of the glomerular cells in the second media for about 2-6 days results in differentiation of the glomerular cells into the engineered podocyte-like cells.

In some embodiments, the engineered podocyte-like cells have increased interdigitating foot processes as compared to the glomerular cells prior to culturing in the first media. In some embodiments, the engineered podocyte-like cells express at least one of F-actin and vimentin. In some embodiments, the engineered podocyte-like cells express at least one of podocin, nephrin, podocalyxin, or synaptopodin.

In some embodiments, the first media comprises the transforming growth factor beta pathway inhibitor, wherein the transforming growth factor beta pathway inhibitor comprises SB431542. In some cases, the concentration of SB431542 is from about 2 μM to 10 μM. In some cases, the concentration of SB431542 is 4 μM. In some embodiments, the first media comprises the Wnt pathway inhibitor, wherein the Wnt pathway inhibitor comprises IWR-1. The method of claim 57, wherein the concentration of IWR-1 is from about 2 μM and 20 μM. In some cases, the concentration of IWR-1 is 2 μM.

In some embodiments, the second media comprises the retinoic acid. In some cases, the concentration of retinoic acid is 0.2 μM. In some embodiments, the second media comprises the ROCK inhibitor, wherein the ROCK inhibitor comprises Y-27632. In some cases, the concentration of Y-27632 is from about 2 μM to about 15 μM. In some cases, the concentration of Y-27632 is 10 μM.

In some embodiments, at least one of the first media and the second media further comprises at least one of heparin, a hormone, and a glycoprotein. In some cases, at least one of the first media and the second media further comprises insulin-transferrin-selenium or a salt thereof. In some cases, least one of the first media and the second media further comprises an antibiotic. In some cases, at least one of the first media and the second media comprises at least one of penicillin and streptomycin.

Also disclosed is a method of making engineered podocyte-like cells, the method comprising: culturing glomerular cells in a media comprising a histone deacetylase inhibitor for at least about 4-8 days, wherein the culturing results in differentiation of the glomerular cells into the engineered podocyte-like cells. In some cases, the histone deacetylase inhibitor comprises a hydroxamic acid or a salt thereof. In some cases, the histone deacetylase inhibitor comprises Panobinostat or a salt thereof. In some cases, the concentration of Panobinostat is from about 50 nM to about 200 nM. In some cases, the concentration of Panobinostat is 50 nM.

In some embodiments, the media further comprises at least one of a hormone and a glycoprotein. In some cases, the media further comprises insulin-transferrin-selenium (ITS). In some cases, the media further comprises an antibiotic. In some cases, the media further comprises at least one of penicillin and streptomycin.

Also disclosed is a method of maintaining engineered podocyte-like cells, the method comprising culturing the engineered podocyte-like cells in media comprising at least one of penicillin-streptomycin, fetal bovine serum, heparin, ascorbic acid, hydrocortisone, rh FGF, rh VEGF, rh EGF, Long R3 IGF, insulin, triiodothyronine, epinephrine, holo-transferrin, and SB431542. In some cases, the concentration of penicillin-streptomycin is 1%. In some cases, the concentration of fetal bovine serum is 2%. In some cases, the concentration of heparin is 1.05 U/mL. In some cases, the concentration of ascorbic acid is 50 μg/mL. In some cases, the concentration of hydrocortisone is 1.15 μg/mL. In some cases, the concentration of rh FGF is 20 ng/mL. In some cases, the concentration of rh VEGF is 5 ng/mL. In some cases, the concentration of rh EGF is 15 ng/mL. In some cases, the concentration of Long R3 IGF is 15 ng/mL. In some cases, the concentration of insulin is 0.125 U/mL. In some cases, the concentration of Triiodothyronine (T3) is 10 nM. In some cases, the concentration of epinephrine is 1 μM. In some cases, the concentration of Holo-transferrin is 5 g/mL. In some cases, the concentration of SB431542 is 4 μM.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1 shows glomerular outgrowth cells (small, elongated/round cells) are transdifferentiated into podocyte-like cells (large, arborized cells) when exposed to the depicted media scheme. Each square represents one day of culture and arrows indicate when media changes occur with a particular media. The cells were cultured for three days in the first media and seven days in the second media.

FIG. 2A-2C show bioengineered kidney functional data and 3D engineered podocyte-like cell culture. FIG. 2A shows pig kidney grafts comprising the engineered podocyte-like cells demonstrated sustained (over 3+ hours) protein retention (indicated by protein values below 100%) and blood cell retention (indicated by low hematocrit %—in the blood). This data shows filtration function of the bioengineered kidney comprising engineered podocyte-like cells when heterotopically implanted in a pig. FIG. 2B shows heterotopically implanted bi-culture (HUVEC and engineered podocyte-like cells) bioengineered kidneys demonstrate filtration function. The protein levels (g/L) and hematocrit (HCT) (%) for serum and urine from a porcine heterotopic kidney transplant model is shown. Urine proteins levels started at 40 g/L and were reduced to about 30 g/L over time. Urine HCT levels were less than about 2% throughout the 5 hours of the experiment. FIG. 2C shows immunofluorescence labeling of recellularized kidneys (recellularized with engineered podocyte-like cells) and repopulation of glomeruli with podocyte-like cells that present a functional podocyte phenotype, expressing podocin (a functional protein of podocytes), and exhibiting primary process formation (a definitive characteristic of podocytes, shown by the arrows).

FIGS. 3A-3F show engineered podocyte-like cells in 2-dimensional cell culture. FIG. 3A shows immunofluorescence labeling of second media-treated glomerular outgrowth cells (e.g., engineered podocyte-like cells) cultured on collagen I for F-actin. FIG. 3B shows immunofluorescence labeling of second media-treated glomerular outgrowth cells (e.g., engineered podocyte-like cells) cultured on collagen I for vimentin. FIG. 3C shows immunofluorescence of second media-treated glomerular outgrowth cells (e.g., engineered podocyte-like cells) displaying prominent nephrin. FIG. 3D shows the change in podocyte gene expression (nephrin (NPHS1), podocin (NPHS2), and synaptopodin (SYNPO) in engineered podocyte-like cells after culturing in the second media as compared to standard R-endothelial media. FIG. 3E and FIG. 3F shows immunofluorescence labeling of engineered podocyte-like cells for vimentin. The arrows indicate where cell secondary processes interact.

FIG. 4 shows a comparison of the expression of podocin, in primary podocytes that were obtained directly from a kidney (left image) as compared to engineered podocyte-like cells (right image), which were created using the methods described herein.

FIG. 5 shows immunofluorescence images of podocalyxin expression in glomerular outgrowth cells prior to (pre-differentiation) and after 6 days of culturing in the second media. After 6 days post-differentiation the glomerular outgrowth cells are differentiated into engineered podocyte-like cells and have increased expression and properly localized expression in podocalyxin. The puncta in the pre-differentiation image shows the stained nucleus of cells with very limited to no expression of podocalyxin. In the 6 days post-differentiation image, podocalyxin is diffuse in the cell membranes of the engineered podocyte-like cells.

FIG. 6A-B shows in vitro functional characterization of bioengineered kidney constructs, such as urine flow rate, normalized protein, and normalized packed cell volume (PCV). FIG. 6A shows normothermic perfusion data from independent HUVEC only (n=5) and bi-culture (n=5) bioengineered kidney constructs are shown. Bi-culture bioengineered kidney constructs comprised HUVEC and engineered podocyte-like cells. PCV (%) readouts are presented as normalized values ([urine value]/[serum value])*100. Error bars denote the mean and standard deviation at each time point. * Indicates p≤0.005 when comparing HUVEC only to bi-culture grafts at each time point with Two-Way ANOVA/Sidak's multiple comparisons test. FIG. 6B shows normothermic perfusion testing of bi-culture (HUVEC and engineered podocyte-like cells) kidney grafts demonstrate filtration function. Bi-culture urine HCT was less than about 15% at 30 min and 60 min. Bi-culture urine protein was less than about 10 g/L at 30 min and 60 min. Graphs depict means and standard deviations from independent HUVEC only (n=3) and bi-culture (n=5) bioengineered kidney constructs. * Indicates p≤0.005 when comparing bi-culture urine values to all other values with Two-Way ANOVA/Sidak's multiple comparisons test.

FIG. 7 shows heterotopic implantation results of co-culture (HUVEC and engineered podocyte-like cells) bioengineered kidney constructs in a porcine heterotopic kidney transplant model. The graphs show urine flow rate, normalized protein, and normalized packed cell volume (PCV). Measures of renal filtration function from three independent acute porcine bi-culture implants (N=1) show improved function with each trial. Protein (mg/dL) and PCV (%) readouts represent normalized values ([urine value]/[serum value])*100.

FIG. 8 shows a summary of the culture strategy for developing a bioengineered kidney using the engineered podocyte-like cells described herein.

FIG. 9 shows a schematic of an exemplary PSM-YoDa differentiation culture schedule, wherein cells are cultured in PSM for 6 days followed by YoDa for 4 days. Media is replaced every 48 hours (indicated by arrows).

FIG. 10 shows the morphology of glomerular outgrowth cells treated with a PSM-YoDa differentiation scheme. The left panel is a brightfield image showing interdigitating foot processes between adjacent differentiated podocytes (circles), a characteristic of mature podocytes forming a filtration barrier. The right panel is an immunofluorescent image of cytoskeletal proteins F-actin (green) and vimentin (red) showing formation of cytoskeletal extensions (arrows) that form foot processes in mature podocytes. Scale bars are 50 microns.

FIG. 11 shows podocyte protein expression following differentiation via the PSM-YoDa differentiation schedule. (Panel A) Immunofluorescence labeling of PSM-YoDa-treated glomerular outgrowth cells for (Panel B) podocin (green) and (Panel C) nephrin (red) demonstrating abundant podocyte protein expression as well as the presence of cells with primary, secondary, and tertiary foot processes (arrow). (Panel D) Flow cytometry demonstrated 75% of cells expressed the podocyte protein nephrin following differentiation.

FIG. 12 shows a schematic of an exemplary Panobinostat differentiation culture schedule, wherein cells are cultured in Panobinostat media for 6 days. Media is replaced every 48 hours (indicated by arrows).

FIG. 13 shows the morphology of glomerular outgrowth cells treated with Panobinostat media. Immunofluorescence staining with cytoskeletal markers vimentin (red) and F-actin (green) show process formation, a characteristic of mature podocytes, after 2 days of Panobinostat treatment (comparing DMEM (A, D) to Panobinostat conditions (B, C, E, F). Changes in morphology were widespread and observed with both 50 nM and 200 nM concentrations of Panobinostat.

FIG. 14 shows podocyte gene expression following Panobinostat differentiation. Podocyte marker gene expression was upregulated after 2 days of treatment with 200 nM Panobinostat relative to control media (RM). 6 days of treatment with 50 nM Panobinostat resulted in higher synaptopodin and podocalyxin gene expression than PSM (gene expression levels relative to RM control media). These results were demonstrated with two different cell lots (GO-HK-220604 and GO-HK-220702). P=passage #.

FIG. 15A-B shows podocyte protein expression following maintenance culture with KCM+SB. Immunofluorescence labeling of (FIG. 15A) nephrin (green), podocin (red) and (FIG. 15B) synaptopodin (green), podocalyxin (red) demonstrating abundant podocyte protein expression in glomerular outgrowth cells differentiated using either PSM (top rows) or YoDa (bottom rows) followed by maintenance culture in KCM+SB. Cells were counterstained with DAPI. Scale bars=100 μm.

### DETAILED DESCRIPTION

### Definitions

Throughout this application, various embodiments may be presented in a range format. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The singular forms “a”, “an”, and “the” are used herein to include plural references unless the context clearly dictates otherwise. Accordingly, unless the contrary is indicated, the numerical parameters set forth in this application are approximations that can vary depending upon the desired properties sought to be obtained.

Unless otherwise indicated, open terms, such as “contain,” “containing,” “include,” “including,”, mean comprising.

The terms “determining”, “measuring”, “evaluating”, “assessing,” “assaying,” and “analyzing” are often used interchangeably herein to refer to forms of measurement and include determining if an element may be present or not (for example, detection), or the amount of an element. These terms can include quantitative and qualitative determinations. Assessing can be alternatively relative or absolute. “Detecting the presence of” includes determining the amount of something present, as well as determining whether it may be present or absent.

The term “substantially” or “essentially” refers to a qualitative condition that exhibits an entire or nearly total range or degree of a feature or characteristic of interest. In some cases, substantially refers to at least about: 70%, 75%, 80%, 85%, 90%, 95%, 99%, 99.9% or 99.99% of the total range or degree of a feature or characteristic of interest. In some cases, the substantially or essentially refers to an amount that can be about 100% of a total amount.

The term “at least partially” refers to a qualitative condition that exhibits a partial range or degree of a feature or characteristic of interest. In some cases, at least partially refers to at least about: 5%, 10%, 20%, 30%, 40%, 50%, 60%, 70%, 75%, 80%, 85%, 90%, 95%, 99%, or 100% of the total range or degree of a feature or characteristic of interest.

As used herein, the term “about” or “approximately” means within an acceptable error range for the particular value as determined by one of ordinary skill in the art, which will depend in part on how the value is measured or determined, e.g., the limitations of the measurement system. For example, “about” means plus or minus 10%, per the practice in the art. Alternatively, “about” means a range of plus or minus 10%, plus or minus 5%, or plus or minus 1% of a given value. Alternatively, particularly with respect to biological systems or processes, the term means within an order of magnitude, within 5-fold, within 4-fold, within 3-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

When used herein, a percentage of a material (e.g., a biological material, an excipient, a compound, and/or an ingredient) of a composition is with respect to a total weight of a composition. In some cases, a percentage of a material of a composition is with respect to a total volume of a composition. In some cases, “Percentage by weight” or “w/w” means ratio of the mass of the specified ingredient verses the mass of the entire composition (e.g., dosage unit).

The term “subject,” “host,” “individual,” and “patient” are as used interchangeably herein to refer to an animal, typically mammalian animals. Any suitable mammal can be administered a composition as described herein or be treated by a method as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Mammals can be any age or at any stage of development, for example a mammal can be neonatal, infant, adolescent, adult or in utero. In some embodiments, a subject is a human. Humans can be more than, or equal to about: 1, 2, 5, 10, 20, 30, 40, 50, 60, 65, 70, 75, 80, 85, 90, 95, 100, 105, 110, 115 or 120 years of age. Humans can be less than about: 1, 2, 5, 10, 20, 30, 40, 50, 60, 65, 70, 75, 80, 85, 90, 95, 100, 105, 110, 115 or about 120 years of age. In some cases, a human can be less than about 18 years of age. In some cases, human can be from about 1 week to about 5 weeks old, 1 month to about 12 months old, from about 1 year to about 20 years, from about 15 years to about 50 years, from about 40 years to about 80 years, or from about 60 years to about 110 years. In some cases, a human can be more than about 18 years of age. A human may be a pediatric subject. A human may be an adult subject. A human can be a child subject. A mammal such as a human can be born a male or a female. In some embodiments, a subject can have or can be suspected of having a disease or condition, such as a kidney disease. The subject can be a patient, such as a patient being treated for a condition or a disease, such as a kidney disease. In some cases, a subject can be a responder to a therapy. In some cases, a subject can be a non-responder to a therapy. A subject can be predisposed to a risk of developing a condition or a disease. A subject can be in remission from a condition or a disease. In some instances, a subject can be healthy. A subject may be a subject in need thereof.

The term “recipient” and their grammatical equivalents as used herein refers to a subject. A recipient may also be in need thereof, such as needing treatment for a disease such as a kidney disease. In some embodiments, a recipient may be in need thereof of a preventative therapy.

A “therapeutically effective amount” refers to an amount of a composition as disclosed herein with or without additional agents that is effective to achieve its intended purpose, for example to treat a disease. Individual patient needs may vary. Generally, the dosage required to provide an effective amount of the composition will vary, depending on the age, health, physical condition, sex, weight, extent of the disease of the recipient, frequency of treatment and the nature and scope of the disease or condition.

As used herein, the terms “treatment” or “treating” refers to a pharmaceutical or other intervention regimen for obtaining beneficial or desired results in the recipient. Beneficial or desired results include but are not limited to a therapeutic benefit and/or a prophylactic benefit. A therapeutic benefit refers to eradication or amelioration of one or more symptoms of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For a prophylactic benefit, a subject at risk of developing a particular disease, or a subject reporting one or more of the physiological symptoms of a disease may undergo a treatment disclosed herein, even though a diagnosis of this disease may not have been made.

As used herein, a “cell” refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell may not originate from a natural organism (e.g. a cell can be synthetically made, sometimes termed an artificial cell). Of particular interest are mammalian cells, from e.g., mammals including test animals and humans. In some cases, a cell is a kidney cell such as a glomerular cell, a podocyte, or a podocyte-like cell. In some cases, a cell is an engineered cell such that the cell was modified by a human to express certain proteins and/or functional characteristics. In some cases, an engineered cell is cultured under artificial conditions such that it expresses certain proteins and/or functional characteristics.

As used herein, a substance is “pure” or “substantially pure” if it is substantially free of other components. The terms “purify,” “purifying” and “purified”, when applied to a cell, can refer to a cell that has been separated from at least some of the components with which it was associated either when initially produced or generated (e.g., whether in nature or in an experimental setting), or during any time after its initial production. A cell or a cell population may be considered purified if it is isolated at or after production, such as from a material or environment containing the cell or cell population, or by passage through culture, and a purified cell or cell population may contain other materials up to at least about 10%, about 20%, about 30%, about 40%, about 50%, about 60%, about 70%, about 80%, about 90%, or above about 90% and still be considered “isolated.” Purified cell and cell populations can be more than at least about 80%, about 85%, about 90%, about 91%, about 92%, about 93%, about 94%, about 95%, about 96%, about 97%, about 98%, about 99%, or more than at least about 99% pure by weight (w/w). In the instance of cell compositions provided herein, the one or more cell types present in the composition can be independently purified from one or more other cells produced and/or present in the material or environment containing the cell type. Cellular compositions and the cellular components thereof are generally purified from an animal or a biological sample.

An isolated cell may have been (1) separated from at least some of the components with which it was associated when initially obtained (whether in nature or in an experimental setting), and/or (2) produced, prepared, purified, and/or manufactured by the hand of man, e.g. using artificial culture conditions such as (but not limited to) culturing in one or more media. Isolated cells can include those cells that are cultured, even if such cultures are not monocultures. Isolated cells can be separated from at least about 10%, about 20%, about 30%, about 40%, about 50%, about 60%, about 70%, about 80%, about 90%, or more of the other components with which they were initially associated. Isolated cells can be more than about 80%, about 85%, about 90%, about 91%, about 92%, about 93%, about 94%, about 95%, about 96%, about 97%, about 98%, about 99%, or more than about 99% pure. A cell population of a biological sample provided herein can comprise one or more cells, which may then be isolated from such sample. Isolated cells may be provided in a form that is not naturally occurring.

The term “decellularized” or “decellularization” as used herein refers to a biostructure (e.g., an isolated organ or portion thereof, or tissue), from which the cellular and tissue content has been reduced or removed leaving behind an intact acellular infra-structure. Organs such as the kidney can be composed of various specialized tissues. Specialized tissue structures of an organ, or parenchyma, can provide specific function associated with the organ. Supporting fibrous network of an isolated organ can be a stroma. Most organs have a stromal framework composed of unspecialized connecting tissue which supports the specialized tissue. The process of decellularization may at least partially remove the cellular portion of the tissue, leaving behind a complex three-dimensional network of extracellular matrix (ECM). An ECM infrastructure may primarily be composed of collagen but can include cytokines, proteoglycans, laminin, fibrillin and other proteins secreted by cells. An at least partially decellularized structure provides a biocompatible substrate onto which different cell populations may be infused or used to be implanted as acellular medical devices that enable cellular infiltration and remodeling following implantation or application. Decellularized biostructures may be rigid, or semi-rigid, having an ability to alter their shapes. Examples of decellularized isolated organs may include, but are not limited to solid organs such as, a heart, a kidney, a liver, a lung, a pancreas, a brain, a bone, a spleen, a gall bladder, a urinary bladder, a uterus, a ureter, and a urethra.

The term “recellularize” or “recellularization” as used herein may refer to an engraftment or distribution of a plurality of cells as described herein onto a decellularized extracellular matrix. A recellularized organ may comprise morphology or activity of a native, non-decellularized organ.

The term “function” and its grammatical equivalents as used herein may refer to a capability of operating, having, or serving an intended purpose. Functional may comprise any percent from baseline to 100% of an intended purpose. For example, functional may comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

The section headings used herein are for organizational purposes only and are not to be construed as limiting the subject matter described.

### Overview

Described herein are engineered podocyte-like cells and methods of making, maintaining, and culturing the same. These cells can be used in the recellularization of a decellularized organ. The recellularization process described herein achieves kidney function by targeting appropriate cells to specific parts of the decellularized extracellular matrix, which can mimic the physiological microstructure of the kidney. A function of the kidney is to filter blood, allowing certain blood components to pass from the blood into the urine, which is completed in a structure of the kidney called the nephron. The filtration process can be mediated by a specialized cell called a podocyte located within a structure called the glomerulus. Because primary podocytes are non-proliferative and terminally differentiated cells, their availability is very limited. This shortfall impedes the ability to produce functioning kidneys for transplantation, which require podocytes to be sourced on the order of 500 million per decellularized kidney to establish the proper ultrafiltration capabilities of the kidney. Thus, there is a need for developing alternatives to primary podocytes.

Disclosed herein are methods of making engineered podocyte-like cells. Also disclosed herein are engineered podocyte-like cells and methods of use, such as recellularization of a decellularized organ with the engineered podocyte-like cells. The engineered podocyte-like cells can be used in a treatment of a disease, such as a kidney disease. In some embodiments, the engineered podocyte-like cell differentiation method described herein leverages the capacity for glomerular outgrowth cells, which can be scaled accordingly, as podocyte precursors to become podocyte-like cells under proper conditions.

### Podocyte-Like Cells

Disclosed herein are engineered podocyte-like cells. As used herein, podocyte-like cells refer to engineered podocyte-like cells. The methods herein can be used to make the engineered podocyte-like cells. Podocyte-like cells can be differentiated from other cells, for example a glomerular cell.

In some embodiments, podocyte-like cells are functionally similar to wild-type podocyte cells, such as primary podocyte cells, and can have one or more of the physiological characteristics of wild-type cells. Podocytes are cells in Bowman's capsule in the kidneys that wrap around capillaries of the glomerulus. Podocytes comprise the epithelial lining of Bowman's capsule and contribute to the filtration of blood. In some cases, podocytes filter blood and retain large molecules such as proteins. Small molecules within the blood such as water, salts, and sugars are filtered as a step in the formation of urine. In some cases, podocytes are specialized epithelial cells that reside in the visceral layer of the capsule. In some cases, the podocyte-like cells described herein filter blood and other liquids. In some cases, the podocyte-like cells can filter and retain large molecules such as proteins but remove small molecules such as water, salts, and sugars. Podocyte-like cells can be used as a replacement to primary podocytes to regain the function in an organ. In some cases, podocyte-like cells can be used in the recellularization of a decellularized organ or portion thereof. In some cases, a podocyte-like cell can be used to replace a primary podocyte cell in a kidney.

In some embodiments, podocytes-like cells can have long foot processes called pedicels. In some instances, the pedicels can wrap around the capillaries and leave slits between them. In some cases, blood or other liquids can be filtered through these slits, each known as a filtration slit, slit diaphragm, or slit pore. In some cases, several proteins, such as nephrin can be required for the pedicels to wrap around the capillaries and function. In some cases, nephrin is a zipper-like protein that forms the slit diaphragm in a podocyte or podocyte-like cells, with spaces between the teeth of the zipper big enough to allow sugar and water through but too small to allow proteins through. In some cases, nephrin defects can be responsible for kidney failure.

In some embodiments, an engineered podocyte-like cell, can comprise increased expression of one or more of: podocin, nephrin, podocalyxin or synaptopodin as compared to a cell. In some instances, a cell can comprise a glomerular cell. In some embodiments, an engineered podocyte-like cell can comprise decreased expression of one or more of: podocin, nephrin, podocalyxin or synaptopodin as compared to a cell such as a primary podocyte. In some cases, an engineered podocyte-like cell can comprise increased expression of one or more of: podocin, nephrin, podocalyxin or synaptopodin as compared to a glomerular cell and decreased expression of one or more of: podocin, nephrin, podocalyxin or synaptopodin as compared to a primary podocyte. In some cases, protein expression, such as the expression of podocin, nephrin, podocalyxin or synaptopodin can be measured by a microscopy assay (e.g., fluorescent microscopy), a Western blot, a dot blot, a functional assay, or any combination thereof. In some cases, a podocyte-like cell can comprise a cytoskeletal organization with multiple extensions as compared to the cytoskeletal organization of a cell, such as a glomerular cell. In some cases, a podocyte-like cell can have more than, less than, or equal to about: 1, 2, 3, 4, 5, 6, 7, 8, 9, or 10 extensions. In some cases, a podocyte-like cell can have about: 1 to about 30 extensions, 1 to about 10 extensions, 5 to about 15 extensions, 3 to about 18 extensions, or 10 to about 20 extensions. In some cases, a podocyte-like cell can have increased gene expression of: NPHS1, NPHS2, SYNPO, or any combination thereof as compared to a cell, such as a glomerular cell. In some cases, increased gene expression can be determined by quantitative reverse-transcriptase PCR, a northern blot, RNA sequencing, or any combination thereof. In some cases, the engineered podocyte-like cells have decreased localization of one or more of: podocin, nephrin, podocalyxin, or synaptopodin as compared to primary podocytes. In some cases, localization can be determined by fluorescent microcopy.

### Methods and Compositions for Making and Maintaining Engineered Podocyte-Like Cells.

Disclosed herein are methods and compositions for making engineered podocyte-like cells. In some embodiments, the method of making engineered podocyte-like cells can comprise culturing a cell in one or more than one media compositions. In some embodiments, the method of making engineered podocyte-like cells can comprise culturing the engineered podocyte-like cells in a first media and a second media. In some embodiments, the method can comprise culturing the engineered podocyte-like cells in a third media. In some cases, the third media is a maintenance media. In some cases, a composition herein can comprise a first media, a second media, or any combination thereof with or without cells, such as engineered podocyte-like cells or glomerular cells. In some embodiments, the cells are cultured in the first media and then transferred to a second media. In some instances, cells can be cultured in the second media and then transferred to the first media. In some cases, cells may be cultured in only the first or only the second media. In some cases, the first media or the second media can be mixed with another media. In some instances, a first media and a second media can be mixed (e.g., in a 1:1 ratio).

In some cases, a glomerular cell can be seeded into a decellularized organ, such as a decellularized kidney, and cultured in a media to be differentiated into an engineered podocyte-like cell. The decellularized kidney may consist of or consist essentially of the extracellular matrix of the native kidney. For example, a glomerular cell can be grafted into the glomeruli of a decellularized kidney (e.g., porcine kidney) and differentiated into an engineered podocyte-like cell using a method described herein. In some cases, a glomerular cell can be grafted into the glomeruli of a decellularized kidney and differentiated into an engineered podocyte-like cell by culturing in a first media and/or a second media. In some cases, a glomerular cell can be differentiated in situ. In some instances, an additional cell population such as a human umbilical vein endothelial cells (HUVEC) can be co-cultured with an engineered podocyte-like cell.

In some embodiments, a method of making engineered podocyte-like cells comprises culturing glomerular cells in a first media for about 2-4 days. In some embodiments, a method of making engineered podocyte-like cells comprises culturing glomerular cells in a first media for about 4-8 days. In some cases, the method comprises culturing the glomerular cells in the first media for about 3, 4, 5, or 6 days. In some cases, the method comprises culturing the glomerular cells in the first media for about 6 days. In some cases, the first media comprises at least one of: a retinoic acid, a salt thereof, a corticosteroid, a salt thereof, a calcitriol, or a salt thereof. In some embodiments, the method comprises transferring the glomerular cells from the first media to a second media, and culturing the glomerular cells in the second media for about 6-12 days. In some cases, the method comprises culturing the glomerular cells in the second media for about 7-10 days or about 7-9 days. In some cases, the method comprises culturing the glomerular cells in the second media for about 7 days. In some cases, the second media comprises at least one of: a SB431542, a salt thereof, an IWR-1-endo, or a salt thereof. In some embodiments, after the glomerular cells are cultured in the second media, the glomerular cells are differentiated into the engineered podocyte-like cells. In some instances, the engineered podocyte-like cells can have increased expression of one or more of: podocin, nephrin, podocalyxin, or synaptopodin as compared to the glomerular cells prior to culturing in the first media, the second media, or both. In some cases, expression of podocin, nephrin, podocalyxin, or synaptopodin can be determined by a fluorescence microscopy, a Western blot, a flow cytometry, or any combination thereof. In some cases, a first media is used before a second media. In some instances, a second media is used before a first media. In some cases, a glomerular cell can comprise a glomerular outgrowth cell, a primary glomerular cell, or an established glomerular cell-line. A glomerular cell can be obtained from any animal, such as a human. In some cases, a glomerular cell can be a pig glomerular cell, a sheep glomerular cell, a goat glomerular cell, s monkey glomerular cell, a cow glomerular cell, a dog glomerular cell, a cat glomerular cell, or a mixture thereof.

In some embodiments, the method can comprise culturing glomerular cells in a first media for about: 1-12 days, 2-8 days, 2-4 days, 3-7 days, 3-4 days, 4-8 days, or 5-6 days. In some embodiments, the method can comprise culturing glomerular cells in a first media for more than, less than, or equal to about: 1 day, 2 days, 3 days, 4 days, 5 days, 6 days, 7 days, 8 days, 9 days, 10 days, 11 days, or 12 days. In some embodiments, the method can comprise culturing glomerular cells in a second media for about: 1-24 days, 4-18 days, 1-8 days, 6-12 days, 5-15 days, 8-16 days, or 9-20 days. In some embodiments, the method can comprise culturing glomerular cells in a second media for more than, less than, or equal to about: 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days or 24 days.

In some embodiments, media of the first media, the second media, or both can be replaced with a fresh media after about 48 hours of cell culturing. In some cases, media of the first media, the second media, or both can be replaced with fresh media after about 1-96 hours of cell culturing. In some cases, media of the first media, the second media, or both can be replaced with fresh media after more than, less than, or equal to about: 6, 12, 24, 48, 72, or 96 hours of cell culturing. In some cases, the fresh media can comprise fresh first media or fresh second media. In some cases, cells can be cultured at any temperature suitable for growth, for example at a temperature of about: 34° C., 35° C., 36° C., 37° C., or 38° C. In some cases, cells can be cultured at a CO₂ concentration of about 2% to about 15% CO2 (v/v).

In some cases, the first media comprises a basal medium, a nutrient mix, an antibiotic, an insulin-transferrin-selenium (ITS), a serum, a retinoic acid, a corticosteroid, a calcitriol, a salt of any of these, or any combination thereof, a salt thereof. In some cases, a basal medium can comprise a Dulbecco's Modified Eagle Medium, a Basal Medium Eagle, a Glasgow Minimum Essential Medium, an Iscove's Modified Dulbecco's Medium, Grace's Insect Medium, a Minimum Essential Medium, an RPMI medium, a McCoy's 5A, or any combination thereof. In some cases, a basal medium can comprise a complex media. In some cases, a nutrient mixture can comprise a F-12, a F-10, a non-essential amino acids solution, or any combination thereof. In some embodiments, a serum can comprise a fetal bovine serum, a horse serum, a calf serum, a rabbit serum, a porcine serum, a goat serum, a human serum, or any combination thereof. In some cases, a media can be a serum free media or a reduced serum media. In some instances, a first media comprises ITS. In some cases, ITS can be used to replace a serum in a media. In some cases, a media can comprise a serum substitute (e.g., serum alternative) or an engineered serum. In some cases, an antibiotic can comprise a penicillin, a streptomycin, a beta lactam, a tetracycline, a trimethoprim-sulfamethoxazole, a lincosamide, a fluoroquinolone, a cephalosporin, a macrolide, an aminoglycoside, amphotericin, chloramphenicol, ampicillin, vancomycin, lincomycin, carbenicillin, gentamicin, neomycin, benzlpenicillin, rifampicin, mitomycin C, kanamycin, erythromycin, fosmidomycin, a salt of any of these, or any combination thereof. In some cases, an antibiotic can comprise a penicillin, or a salt thereof and a streptomycin, or a salt thereof. In some cases, a first media can comprise a balanced salt solution such as phosphate-buffered saline, Dulbecco's phosphate-buffered saline, Hanks' balanced salt solution, Earle's balanced salts, or any combination thereof. In some cases, a first media can comprise an endothelial growth media. In some cases, an endothelial growth media can comprise Endothelial Cell Growth Base Media supplemented with one or more of fetal bovine serum, ascorbic acid, hydrocortisone, Fibroblast growth factor (FGF), Vascular endothelial growth factor (VEGF), Epidermal growth factor (EGF), R3 IGF, heparin, acetic acid, and/or an antibiotic.

In some embodiments, a media herein can comprise an antibiotic in an amount (weight/weight or volume/volume) of more than, less than, or equal to about: 0.1%, 0.5%, 1%, 2%, 3%, 4%, 5%, 6%, 7%, 8%, 9%, or 10%. In some cases, a media herein can comprise an antibiotic in an amount (weight/weight or volume/volume) of about: 0.1% to 1%, 0.1% to 10%, 1% to 10% or 3% to 8%. In some embodiments, a media herein can comprise an serum in an amount (weight/weight or volume/volume) of more than, less than, or equal to about: 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%. In some cases, a media herein can comprise a serum in an amount (weight/weight or volume/volume) of about: 0.1% to 60%, 1% to 10%, 5% to 25%, 10% to 20%, 15% to 40% or 25% to 50%, or 30% to 60%. In some embodiments, a media herein can comprise an ITS in an amount (weight/weight or volume/volume) of more than, less than, or equal to about: 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%. In some cases, a media herein can comprise an ITS in an amount (weight/weight or volume/volume) of about: 0.1% to 60%, 1% to 10%, 5% to 25%, 10% to 20%, 15% to 40% or 25% to 50%, or 30% to 60%. In some cases, a media herein can comprise an ITS in an amount (weight/weight or volume/volume) of about: 0.5×, 1×, 2×, 3×, 4×, or 5×.

In some embodiments, the first media comprises at least one of: a retinoic acid, a salt thereof, a corticosteroid, a salt thereof, a calcitriol, or a salt thereof. In some cases, the first media comprises at least one of: a transforming growth factor beta pathway inhibitor or a salt thereof, and a Wnt pathway inhibitor or a salt thereof. In some cases, a media comprises Panobinostat.

In some cases, a first media can comprise a retinoic acid. In some cases, retinoic acid can exert a pleotropic cellular effect, such as induction of cell differentiation while inhibiting proliferation and inflammation. In some cases, a retinoic acid can be used to protect and/or differentiate podocytes. In some cases, a retinoic acid comprises vitamin A, a derivative thereof, or a salt of any of these. In some cases, a retinoic acid comprises an all-trans-retinoic acid. In some cases, a retinoic acid comprises an isomer of retinoic acid such as 12-cis or 9-cis-retinoic acid. In some cases, a retinoic acid comprises a precursor of retinoic acid such as retinol, a derivative thereof, or a salt thereof. In some cases, a media can comprise a retinoic acid or a salt thereof in a concentration of about 0.1 μM to about 1 mM. In some cases, a media can comprise a retinoic acid or a salt thereof in a concentration of about: 0.1 μM to 10 μM, 1 μM to 10 μM, 0.1 μM to 1 μM, 1 μM to 5 μM, 5 μM to 25 μM, 10 μM to 100 μM, 50 μM to 500 μM, 250 μM to 1000 μM, 0.1 mM to 1 mM, or 1 mM to about 10 mM. In some cases, a media comprises a retinoic acid or a salt thereof in a concentration of more than, less than, or equal to about: 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 M, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 1000 μM, 1 mM, 2 mM, 3 mM, 4 mM, 5 mM, 6 mM, 7 mM, 8 mM, 9 mM, or 10 mM.

In some cases, a first media can comprise a corticosteroid or a salt thereof. In some cases, a glomerular podocyte can comprise a functional receptors for a corticosteroid. In some cases, a corticosteroid can exhibit a clinical effect and rescue podocyte function in a cell. Corticosteroids like dexamethasone can increase the stability of the cytoskeletal protein F-actin in podocytes and/or at least partially inhibit podocyte apoptosis. In some cases, a corticosteroid can comprise dexamethasone or a salt thereof. In some cases, a corticosteroid can comprise a cortisone, a prednisone, a prednisolone, a methylprednisolone, a dexamethasone, a betamethasone, a hydrocortisone, a bethamethasone, a triamcinolone, a salt of any of these, or any combination thereof. In some cases, a corticosteroid can comprise a synthetic corticosteroid. In some cases, a media can comprise a corticosteroid, or a salt thereof in a concentration of about 100 nM to about 10 mM. In some cases, a media can comprise a corticosteroid, or a salt thereof in a concentration of about: 10 nM to 1000 nM, 50 nM to 250 nM, 75 nM to 500 nM, 100 nM to 1000 nM, 0.1 μM to 1 μM, 1 μM to 5 μM, 5 μM to 25 μM, 10 μM to 100 μM, 50 μM to 500 μM, 250 μM to 1000 μM, 0.1 mM to 1 mM, or 1 mM to about 10 mM. In some cases, a media comprises a corticosteroid, or a salt thereof in a concentration of more than, less than, or equal to about: 5 nM, 10 nM, 20 nM, 50 nM, 70 nM, 80 nM, 90 nM, 100 nM, 150 nM, 250 nM, 0.1 μM, 0.2 M, 0.3 μM, 0.4 μM, 0.5 μM, 0.6 μM, 0.7 μM, 0.8 μM, 0.9 μM, 1 μM, 10 μM, 50 μM, 100 μM, 500 μM, 1 mM, or 10 mM.

In some cases, a first media can comprise a calcitriol or a salt thereof. Calcitriol, the biologically active form of vitamin D, can exert its biological effects by activating the vitamin D receptor. Calcitriol can be effective in reducing podocyte damage and/or promoting podocyte gene expression. In some cases, calcitriol can comprise vitamin D, a derivative thereof, or a salt thereof. In some cases, calcitriol can comprise 1,25-dihydroxycholecalciferol. In some cases, calcitriol can comprise vitamin D3 (e.g., cholecalciferol) and vitamin D2 (e.g., ergocalciferol), vitamin D4 (e.g., 22-dihydroergocalciferol), vitamin D5 (e.g., sitocalciferol) a derivative of any of these, a salt of any of these, or any combination thereof. In some cases, a calcitriol comprises a precursor of calcitriol. In some cases, a media can comprise a calcitriol, or a salt thereof in a concentration of about 1 nM to about 300 nM. In some cases, a media can comprise a calcitriol, or a salt thereof in a concentration of about: 1 nM to 1000 nM, 10 nM to 300 nM, 50 nM to 250 nM, 75 nM to 500 nM, 30 nM to 200 nM, 50 nM to 275 nM, 100 nM to 400 nM, 100 nM to 1000 nM, or 0.1 μM to 1 μM. In some cases, a media comprises a calcitriol, or a salt thereof in a concentration of more than, less than, or equal to about: 1 nM, 5 nM, 10 nM, 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 500 nM, 0.1 μM, 0.2 μM, 0.3 μM, 0.4 μM, 0.5 μM, 0.6 μM, 0.7 μM, 0.8 μM, 0.9 μM, 1 μM, 10 μM, 50 μM, 100 μM, 500 μM, 1 mM, or 10 mM.

In some cases, a second media comprises a basal medium, a nutrient mix, an antibiotic, an insulin-transferrin-selenium (ITS), a fetal bovine serum, a SB431542, an IWR-1-endo (also referred to herein as IWR-1), a salt of any of these, or any combination thereof, a salt thereof. In some cases, a second media comprises at least one of: a retinoic acid or a salt thereof, and a ROCK inhibitor or a salt thereof. In some cases, a basal medium can comprise a Dulbecco's Modified Eagle Medium, a Basal Medium Eagle, a Glasgow Minimum Essential Medium, an Iscove's Modified Dulbecco's Medium, Grace's Insect Medium, a Minimum Essential Medium, an RPMI medium, a McCoy's 5A, or any combination thereof. In some cases, a basal medium can comprise a complex media. In some cases, a nutrient mixture can comprise a F-12, a F-10, a non-essential amino acids solution, or any combination thereof. In some embodiments, a serum can comprise a fetal bovine serum, a horse serum, a calf serum, a rabbit serum, a porcine serum, a goat serum, a human serum, or any combination thereof. In some cases, a media can be a serum free media or a reduced serum media. In some instances, a second media comprises ITS. In some cases, ITS can be used to replace a serum in a media. In some cases, a media can comprise a serum substitute (e.g., serum alternative) or an engineered serum. In some cases, an antibiotic can comprise a penicillin, a streptomycin, a beta lactam, a tetracycline, a trimethoprim-sulfamethoxazole, a lincosamide, a fluoroquinolone, a cephalosporin, a macrolide, an aminoglycoside, amphotericin, chloramphenicol, ampicillin, vancomycin, lincomycin, carbenicillin, gentamicin, neomycin, benzlpenicillin, rifampicin, mitomycin C, kanamycin, erythromycin, fosmidomycin, a salt of any of these, or any combination thereof. In some cases, an antibiotic can comprise a penicillin, or a salt thereof and a streptomycin, or a salt thereof. In some cases, a media such as a second media, can comprise a balanced salt solution such as phosphate-buffered saline, Dulbecco's phosphate-buffered saline, Hanks' balanced salt solution, Earle's balanced salts, or any combination thereof.

In some embodiments, a second media can comprise at least one of: a SB431542, a salt thereof, an IWR-1-endo, or a salt thereof.

In some cases, a first media and/or a second media can comprise a SB431542 or a salt thereof. SB431542 can be a potent transforming growth factor beta pathway inhibitor. In podocytes, SB431542 has been shown to promote podocyte function and/or protect podocytes from injury. In some cases, SB431542 can comprise the formula C₂₂H₁₆N₄O₃. In some cases, SB431542 can comprise the CAS number 301836-41-9. In some cases, SB431542 can comprise a derivative of SB431542 or a salt thereof. In some cases, a second media can comprise a growth factor beta pathway inhibitor. In some cases, a media can comprise a SB431542 or a salt thereof in a concentration of about 0.1 μM to about 100 μM. In some cases, a media can comprise a SB431542 or a salt thereof in a concentration of about: 0.1 μM to 100 μM, 1 μM to 10 μM, 1 μM to 15 μM, 5 μM to 15 μM, 0.1 μM to 1 μM, 1 μM to 5 μM, 5 μM to 25 μM, 10 μM to 100 μM, 50 μM to 75 μM, or 80 μM to 100 μM. In some cases, a media comprises a SB431542 or a salt thereof in a concentration of more than, less than, or equal to about: 0.1 μM, 0.2 μM, 0.3 μM, 0.4 μM, 0.5 M, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 60 M, 70 μM, 80 μM, 90 μM, 100 μM, 110 μM, 120 μM, 130 μM, 140 μM, 150 μM, 160 μM, 170 μM, 180 μM, 190 μM, or 200 μM.

In some cases, a first media and/or a second media can comprise a Wnt pathway inhibitor, such as IWR-1-endo or a salt thereof. In some cases, IWR-1 is a Wnt pathway inhibitor that can promote podocyte differentiation. In some cases, a second media can comprise a Wnt pathway inhibitor. In some cases, IWR-1-endo can comprise IWR-1. In some cases, IWR-1 can comprise IWR-1-endo. In some cases, IWR-1-endo can comprise the formula C₂₅H₁₉N₃O₃. In some cases, IWR-1-endo can comprise the CAS number 1127442-82-3. In some cases, IWR-1-endo can comprise a derivative of IWR-1-endo or a salt thereof. In some cases, a media can comprise a IWR-1-endo or a salt thereof in a concentration of about 0.1 μM to about 100 μM. In some cases, a media can comprise a IWR-1-endo or a salt thereof in a concentration of about: 0.1 μM to 100 μM, 1 μM to 10 μM, 1 μM to 15 μM, 5 μM to 15 μM, 0.1 μM to 1 μM, 1 μM to 5 μM, 5 μM to 25 μM, 10 μM to 100 μM, 50 μM to 75 μM, or 80 μM to 100 μM. In some cases, a media comprises a IWR-1-endo or a salt thereof in a concentration of more than, less than, or equal to about: 0.1 μM, 0.2 μM, 0.3 μM, 0.4 μM, 0.5 M, 0.6 μM, 0.7 UM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 M, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, or 200 μM.

In some cases, the first media and/or the second media can comprise a ROCK inhibitor or a salt thereof. In some cases, the ROCK inhibitor comprises Y-27632. In some cases, the Y-27632 comprises the formula C₁₄H₂₁N₃O. In some cases, Y-27632 can comprise the CAS number 129830-38-2. In some cases, Y-27632 can comprise a derivative of Y-27632 or a salt thereof. In some cases, a media can comprise a Y-27632 or a salt thereof in a concentration of about 0.1 μM to about 100 μM. In some cases, a media can comprise a Y-27632 or a salt thereof in a concentration of about: 0.1 μM to 100 μM, 1 μM to 10 μM, 1 μM to 15 μM, 5 μM to 15 μM, 0.1 μM to 1 μM, 1 μM to 5 μM, 5 μM to 25 μM, 10 μM to 100 μM, 50 μM to 75 μM, or 80 μM to 100 μM. In some cases, a media comprises a Y-27632 or a salt thereof in a concentration of more than, less than, or equal to about: 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 60 μM, 70 μM, 80 μM, 90 μM, 100 μM, 110 μM, 120 μM, 130 μM, 140 μM, 150 M, 160 μM, 170 μM, 180 μM, 190 M, or 200 μM.

In some cases, a media comprises Panobinostat or a salt thereof. In some cases, the Panobinostat comprises the formula C₂₁H₂₃N₃O₂. In some cases, Panobinostat can comprise the CAS number 404950-80-7. In some cases, Panobinostat can comprise a derivative of Panobinostat or a salt thereof. In some cases, a media can comprise a Panobinostat or a salt thereof in a concentration of about 0.1 nM to about 200 nM. In some cases, a media can comprise a Panobinostat or a salt thereof in a concentration of about: 0.1 nM to 100 nM, 1 nM to 10 nM, 1 nM to 15 nM, 5 nM to 15 nM, 0.1 nM to 1 nM, 1 nM to 5 nM, 5 nM to 25 nM, 10 nM to 100 nM, 50 nM to 75 nM, or 80 nM to 100 nM. In some cases, a media comprises a Panobinostat or a salt thereof in a concentration of more than, less than, or equal to about: 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, or 200 nM.

In some cases, a media comprises hydrocortisone. In some cases, the hydrocortisone comprises the formula C₂₁H₃₀O₅. In some cases, hydrocortisone can comprise the CAS number 50-23-7. In some cases, hydrocortisone can comprise a derivative of hydrocortisone or a salt thereof. In some cases, a media can comprise a hydrocortisone or a salt thereof in a concentration of about 0.5 μg/mL to 5 μg/mL. In some cases, a media can comprise a hydrocortisone or a salt thereof in a concentration of about: 0.5 μg/mL, 0.6 μg/mL, 0.7 μg/mL, 0.8 μg/mL, 0.9 μg/mL, 1 μg/mL, 1.5 μg/mL, 2 μg/mL, 2.5 μg/mL, 3 μg/mL, 3.5 μg/mL, 4 μg/mL, 4.5 μg/mL, or 5 μg/mL. In some cases, a media can comprise about 1.15 μg/mL hydrocortisone.

In some cases, a media comprises a recombinant human fibroblast growth factor (rh FGF) or a variant thereof. In some cases, the rh FGF comprises the amino acid sequence set forth in SEQ ID NO: 1 or a variant thereof. In some cases, the rh FGF comprises an E. coli-derived human FGF basic, Pro143-Ser288, with an N-terminal Ala, Accession #P09038. In some cases, rh FGF can comprise a derivative of rh FGF or a salt thereof. In some cases, a media can comprise a rh FGF or a salt thereof in a concentration of about 1 ng/mL to 100 ng/mL. In some cases, a media can comprise a rh FGF or a variant or a salt thereof in a concentration of about: 1 ng/mL, 2 ng/ml, 3 ng/mL, 4 ng/mL, 5 ng/mL, 6 ng/ml, 7 ng/mL, 8 ng/ml, 9 ng/ml, 10 ng/mL, 15 ng/mL, 20 ng/ml, 25 ng/ml, 30 ng/mL, 35 ng/mL, 40 ng/mL, 45 ng/mL, 50 ng/mL, 55 ng/mL, 60 ng/mL, 65 ng/mL, 70 ng/ml, 75 ng/mL, 80 ng/mL, 85 ng/ml, 90 ng/ml, 95 ng/ml, or 100 ng/mL.

In some cases, a media comprises recombinant human vascular endothelial growth factor (rh VEGF) or a variant thereof. In some cases, the rh VEGF comprises the amino acid sequence set forth in SEQ ID NO: 2 or a variant thereof. In some cases, the rh VEGF comprises an Sf21 (baculorvirus)-derived human VEGF 165, Ala27-Arg191, Accession #NP_001165097. In some cases, rh VEGF can comprise a derivative of rh VEGF or a salt thereof. In some cases, a media can comprise a rh VEGF or a salt thereof in a concentration of about 1 ng/ml to 100 ng/ml. In some cases, a media can comprise a rh VEGF or a variant or a salt thereof in a concentration of about: 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/mL, 20 ng/mL, 25 ng/ml, 30 ng/mL, 35 ng/ml, 40 ng/ml, 45 ng/ml, 50 ng/ml, 55 ng/ml, 60 ng/ml, 65 ng/ml, 70 ng/ml, 75 ng/ml, 80 ng/mL, 85 ng/mL, 90 ng/mL, 95 ng/ml, or 100 ng/mL.

In some cases, a media comprises a recombinant human epidermal growth factor (rh EGF) or a variant thereof. In some cases, the rh EGF comprises the amino acid sequence set forth in SEQ ID NO: 3 or a variant thereof. In some cases, the rh EGF comprises an E. coli-derived human EGF protein, Asn971-Arg1023, with an N-terminal Met, Accession #P01133. In some cases, rh EGF can comprise a derivative of rh EGF or a salt thereof. In some cases, a media can comprise a rh EGF or a salt thereof in a concentration of about 1 ng/mL to 100 ng/mL. In some cases, a media can comprise a rh EGF or a variant or a salt thereof in a concentration of about: 1 ng/mL, 2 ng/mL, 3 ng/ml, 4 ng/ml, 5 ng/mL, 6 ng/ml, 7 ng/ml, 8 ng/ml, 9 ng/ml, 10 ng/ml, 15 ng/ml, 20 ng/mL, 25 ng/mL, 30 ng/mL, 35 ng/mL, 40 ng/mL, 45 ng/mL, 50 ng/mL, 55 ng/ml, 60 ng/ml, 65 ng/mL, 70 ng/ml, 75 ng/ml, 80 ng/ml, 85 ng/ml, 90 ng/ml, 95 ng/ml, or 100 ng/mL.

In some cases, a media comprises a recombinant human long R3 insulin-like growth factor (Long R3 IGF) or a variant thereof. In some cases, the Long R3 IGF comprises the amino acid sequence set forth in SEQ ID NO: 4 or a variant thereof. In some cases, a Long R3 IGF comprises an E. coli-derived human IGF-I, Gly49-Ala118 (Glu51Arg), N-terminus MFPAMPLSSLFVN (SEQ ID NO: 5), Accession #P05019.1. In some cases, Long R3 IGF can comprise a derivative of Long R3 IGF or a salt thereof. In some cases, a media can comprise a Long R3 IGF or a salt thereof in a concentration of about 1 ng/mL to 100 ng/mL. In some cases, a media can comprise a Long R3 IGF or a variant or a salt thereof in a concentration of about: 1 ng/ml, 2 ng/ml, 3 ng/ml, 4 ng/ml, 5 ng/mL, 6 ng/ml, 7 ng/mL, 8 ng/ml, 9 ng/mL, 10 ng/ml, 15 ng/ml, 20 ng/ml, 25 ng/ml, 30 ng/mL, 35 ng/mL, 40 ng/ml, 45 ng/mL, 50 ng/mL, 55 ng/mL, 60 ng/ml, 65 ng/ml, 70 ng/mL, 75 ng/mL, 80 ng/mL, 85 ng/ml, 90 ng/ml, 95 ng/ml, or 100 ng/ml.

In some cases, a media comprises insulin from bovine pancreas. In some cases, the insulin comprises the formula C₂₅₄H₃₇₇N₆₅O₇₅S₆. In some cases, insulin can comprise the CAS number 11070-73-8. In some cases, insulin can comprise a derivative of insulin or a salt thereof. In some cases, a media can comprise a insulin or a salt thereof in a concentration of about 0.01 U/mL to 10 U/mL. In some cases, a media can comprise an insulin or a salt thereof in a concentration of about: 0.01 U/mL, 0.02 U/mL, 0.03 U/mL, 0.04 U/mL, 0.05 U/mL, 0.06 U/mL, 0.07 U/mL, 0.08 U/mL, 0.09 U/mL, 0.1 U/mL, 0.125 U/mL, 0.150 U/mL, 0.175 U/mL, 0.2 U/mL, 0.225 U/mL, 0.250 U/mL, 0.275 U/mL, 0.3 U/mL, 0.325 U/mL, 0.350 U/mL, 0.375 U/mL, 0.4 U/mL, 0.425 U/mL, 0.450 U/mL, 0.475 U/mL, 0.5 U/mL, 0.525 U/mL, 0.550 U/mL, 0.575 U/mL, 0.6 U/mL, 0.625 U/mL, 0.650 U/mL, 0.675 U/mL, 0.7 U/mL, 0.725 U/mL, 0.75 U/mL, 0.775 U/mL, 0.8 U/mL, 0.825 U/mL, 0.850 U/mL, 0.875 U/mL, 0.9 U/mL, 0.925 U/mL, 0.950 U/mL, 0.975 U/mL, 1 U/mL, 1.5 U/mL, 2 U/mL, 2.5 U/mL, 3 U/mL, 3.5 U/mL, 4 U/mL, 4.5 U/mL, 5 U/mL, 5.5 U/mL, 6 U/mL, 6.5 U/mL, 7 U/mL, 7.5 U/mL, 8 U/mL, 8.5 U/mL, 9 U/mL, 9.5 U/mL, or 10 U/mL.

In some cases, a media comprises triiodothyronine. In some cases, the triiodothyronine comprises the formula C₁₅H₁₂I₃NO₄. In some cases, triiodothyronine can comprise the CAS number 6893-2-3. In some cases, triiodothyronine can comprise a derivative of triiodothyronine or a salt thereof. In some cases, a media can comprise a triiodothyronine or a salt thereof in a concentration of about 0.1 nM to 100 nM. In some cases, a media can comprise a triiodothyronine or a salt thereof in a concentration of about: 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 60 nM, 70 nM, 80 nM, 90 nM, or 100 nM.

In some cases, a media comprises epinephrine or a salt thereof. In some cases, the epinephrine comprises the formula C₉H₁₃NO₃. In some cases, epinephrine can comprise the CAS number 329-63-5. In some cases, epinephrine can comprise a derivative of epinephrine or a salt thereof. In some cases, a media can comprise an epinephrine or a salt thereof in a concentration of about 0.1 μM to 10 μM. In some cases, a media can comprise an epinephrine or a salt thereof in a concentration of about: 0.1 μM, 0.2 μM, 0.3 μM, 0.4 μM, 0.5 μM, 0.6 μM, 0.7 μM, 0.8 μM, 0.9μ, 1μ, 1.5μ, 2μ, 2.5μ, 3μ, 3.5μ, 4μ, 4.5μ, 5μ, 5.5μ, 6μ, 6.5μ, 7 μM, 7.5 μM, 8 μM, 8.5 μM, 9 μM, 9.5 μM, or 10 μM.

In some cases, a media comprises a holo-transferrin or a variant thereof. In some cases, the holo-transferrin comprises the CAS number 11096-37-0. In some cases, a media can comprise an holo-transferrin or a variant or a salt thereof in a concentration of about 0.1 μg/mL to 100 μg/mL. In some cases, a media can comprise a holo-transferrin or a variant or a salt thereof in a concentration of about: 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 g/mL, 1 μg/mL, 2 μg/mL, 3 μg/mL, 4 μg/mL, 5 g/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 60 μg/mL, 70 μg/mL, 80 μg/mL, 90 μg/mL, or 100 μg/mL.

In some embodiments, a first, second, or third media, such as for example a maintenance media, comprises at least one of: penicillin-streptomycin, fetal bovine serum, heparin, ascorbic acid, hydrocortisone, rh FGF, rh VEGF, rh EGF, Long R3 IGF, insulin, triiodothyronine, epinephrine, holo-transferrin, and SB431542.

### Decellularized Organs

Disclosed herein are at least partially recellularized kidneys or portions thereof, prepared from a decellularized extracellular matrix. Methods for decellularization and recellularization are disclosed in U.S. patents, including U.S. Pat. Nos. 8,470,520, 10,233,420, and 10,220,056, which are incorporated herein by reference in their entirety.

In some cases, the initial step in decellularizing an organ or tissue, such as a kidney, is to cannulate the organ or tissue, if possible. The vessels, ducts, and/or cavities of an organ or tissue can be cannulated using common methods and materials. The next step in decellularizing an organ or tissue can be to perfuse the cannulated organ or tissue with a cellular disruption medium. Perfusion through an organ can be multi-directional (e.g., antegrade and retrograde). A cellular disruption medium can be delivered by an infusion or roller pump or by a constant hydrostatic pressure.

One or more cellular disruption media can be used to decellularize an organ or tissue. A cellular disruption medium generally includes at least one detergent such as SDS, PEG, or Triton X. A cellular disruption medium can include water such that the medium is osmotically incompatible with the cells. Alternatively, a cellular disruption medium can include a buffer (e.g., PBS) for osmotic compatibility with the cells. Cellular disruption media also can include enzymes such as, without limitation, one or more collagenases, one or more dispases, one or more DNases, or a protease such as trypsin. In some instances, cellular disruption media also or alternatively can include inhibitors of one or more enzymes (e.g., protease inhibitors, nuclease inhibitors, and/or collegenase inhibitors).

In certain embodiments, a cannulated organ or tissue can be perfused sequentially with two different cellular disruption media. For example, the first cellular disruption medium can include an anionic detergent such as SDS and the second cellular disruption medium can include an ionic detergent such as Triton X. Following perfusion with at least one cellular disruption medium, a cannulated organ or tissue can be perfused, for example, with wash solutions and/or solutions containing one or more enzymes such as those disclosed herein. Alternating the direction of perfusion (e.g., antegrade and retrograde) can help to effectively decellularize the entire organ or tissue. Decellularization as described herein essentially decellularizes the organ from the inside out, resulting in very little damage to the ECM. An organ or tissue can be decellularized at a suitable temperature between 4 and 40° C. Depending upon the size and weight of an organ or tissue and the particular detergent(s) and concentration of detergent(s) in the cellular disruption medium, an organ or tissue generally is perfused from about 0.1 to about 12 hours per gram of solid organ or tissue with cellular disruption medium. Including washes, an organ may be perfused for up to about 12 to about 72 hours per gram of tissue. Perfusion generally is adjusted to physiologic conditions including pulsatile flow, rate and pressure. In some aspects, a cellular disruption solution is a solution that can comprise at least one detergent. A detergent can be an amphipathic molecule, that can contain both a nonpolar “tail” having aliphatic or aromatic character and a polar “head”. Ionic character of the polar head group can form the basis for broad classification of detergents; they may be ionic (charged, either anionic or cationic), nonionic (uncharged), or zwitterionic (having both positively and negatively charged groups but with a net charge of zero). In some aspects, detergents can be denaturing or non-denaturing with respect to protein structure. Denaturing detergents can be anionic such as sodium dodecyl sulfate (SDS) or cationic such as ethyl trimethyl ammonium bromide (ETMAB). These detergents can disrupt membranes and denature proteins by breaking protein-protein interactions. Non-denaturing detergents can be divided into nonionic detergents such as Triton X-100, NP40, Tween, bile salts such as cholate, and zwitterionic detergents such as CHAPS.

In some cases, a decellularized organ or tissue consists essentially of the extracellular matrix (ECM) component of all or most regions of the organ or tissue, including ECM components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue. The morphology and architecture of the ECM can be examined visually and/or histologically.

One or more compounds can be applied in or on a decellularized organ or tissue to, for example, to preserve the decellularized organ, or to prepare the decellularized organ or tissue for recellularization and/or to assist or stimulate cells during the recellularization process. Such compounds include, but are not limited to, one or more growth factors (e.g., VEGF, DKK-1, FGF, bFGF, PDGF, HGF, BMP-1, BMP-4, SDF-1, IGF, and HGF), immune modulating agents (e.g., cytokines, glucocorticoids, IL2R antagonist, leucotriene antagonists, including but not limited to antibody therapy, use of stem cells to modulate the immune response, etc.), and/or factors that modify the coagulation cascade (e.g., aspirin, heparin-binding proteins, and heparin). In addition, a decellularized organ or tissue can be further treated with, for example, irradiation (e.g., UV, gamma) to reduce or eliminate the presence of any type of microorganism remaining on or in a decellularized organ or tissue.

In some aspects, perfusion decellularization comprises cannulating an organ or portion thereof. In some aspects, at least one cannulation is introduced to an organ or portion thereof. In some aspects, at least two cannulations are introduced to an organ or portion thereof. In some aspects, from about 1, 2, 3, 4, 5, 6, 7, 8, 9, or up to 10 cannulations are introduced to an organ or portion thereof. In some cases, a cannula can be a part of a cannulation system. A cannulation system can comprise a size-appropriate hollow tubing for introducing into a vessel, duct, cavity, or any combination thereof of an organ or tissue. Typically, at least one vessel, duct, and/or cavity is cannulated in an organ. A perfusion apparatus or cannulation system can include a holding container for solutions (e.g., a cellular disruption medium) and a mechanism for moving the liquid through the organ (e.g., a pump, air pressure, gravity) via the one or more cannulae. The sterility of an organ or tissue during decellularization and/or recellularization can be maintained using a variety of techniques known in the art such as controlling and filtering the air flow and/or perfusing with, for example, antibiotics, anti-fungals or other anti-microbials to prevent the growth of unwanted microorganisms. In some aspects, a system as described herein can possess the ability to monitor certain perfusion characteristics (e.g., pressure, volume, flow pattern, temperature, gases, pH), mechanical forces (e.g., ventricular wall motion and stress), and electrical stimulation (e.g., pacing). In some aspects, a vascular bed can change over the course of decellularization and recellularization (e.g., vascular resistance, volume), a pressure-regulated perfusion apparatus or cannulation system can be advantageous to avoid or reduce fluctuations. The effectiveness of perfusion can be evaluated in the effluent and in tissue sections. Perfusion volume, flow pattern, temperature, partial O2 and CO2 pressures and pH can be monitored using standard methods. In some aspects, sensors can be used to monitor the system (e.g., bioreactor) and/or the organ or tissue. Sonomicrometry, micromanometry, and/or conductance measurements can be used to acquire pressure-volume or preload recruitable stroke work information relative to myocardial wall motion and performance. For example, sensors can be used to monitor the pressure of a liquid moving through a cannulated organ or tissue; the ambient temperature in the system and/or the temperature of the organ or tissue; the pH and/or the rate of flow of a liquid moving through the cannulated organ or tissue; and/or the biological activity of a recellularizing organ or tissue. In addition to having sensors for monitoring such features, a system for decellularizing and/or recellularizing an organ or tissue also can include means for maintaining or adjusting such features. Means for maintaining or adjusting such features can include components such as a thermometer, a thermostat, electrodes, pressure sensors, overflow valves, valves for changing the rate of flow of a liquid, valves for opening and closing fluid connections to solutions used for changing the pH of a solution, a balloon, an external pacemaker, and/or a compliance chamber. To help ensure stable conditions (e.g., temperature), the chambers, reservoirs, and tubings can be water-jacketed. some aspects, the cannulation occurs at a cavity, vessel, duct, or combination thereof. In some aspects, from about 1 to 3, from about 1 to 5, from about 2 to 3, from about 2 to 5, from about 1 to 8 solutions can be utilized for organ perfusion. In some aspects, a solution is perfused at least two times. In some aspects, a solution is perfused at least 3, 4, 5, 6, 7, 8, 9, or up to 10 times through the organ or portion thereof. Various solutions and mediums can be employed during recelluarization. In some aspects, a solution can be selected from the group comprising: cellular disruption solutions, washing solutions, disinfecting solutions, or combinations thereof.

In some aspects, a washing solution may be utilized during decellularization. A washing solution may be utilized to remove residual solutions such as cellular disruption solutions from an organ or portion thereof as well as residual cellular components, enzymes, or combinations thereof. Suitable washing solutions may comprise water, filtered water, Phosphate buffered saline (PBS), and combinations thereof. PBS can maintain a constant pH and the osmolarity of cells. In some cases, the pH of most biological materials falls from about 6.8 to about 7.6.

In some aspects, a disinfecting solution may be utilized during decellularization. A disinfecting solution may comprise any number of agents such as antibiotics, disinfectants, or combinations thereof. In some aspects, an antibiotic that can be used in a decellularization solution can be selected from the group comprising: actinomycin, ampicillin, carbenicillin, cefotaxime, fosmidomycin, gentamicin, kanamycin, neomycin, amphotericin, penicillin, polymyxin, streptomycin, broad selection antibiotic, and combinations thereof. Any concentration of antibiotic may be introduced in a disinfecting solution.

In some aspects, a system, such as a system for generating an organ or portion thereof or tissue may be controlled by a computer-readable storage medium in combination with a programmable processor (e.g., a computer-readable storage medium as used herein has instructions stored thereon for causing a programmable processor to perform particular steps). For example, such a storage medium, in combination with a programmable processor, may receive and process information from one or more of the sensors. Such a storage medium in conjunction with a programmable processor also can transmit information and instructions back to the bioreactor and/or the organ or tissue. In some aspects, an organ or tissue undergoing recellularization may be monitored for biological activity. Biological activity can be that of the organ or portion thereof or tissue itself such as for kidney tissue, electrical activity, mechanical activity, mechanical pressure, contractility, and/or wall stress of the organ or tissue. In addition, the biological activity of cells attached or engrafted on to the organ or portion thereof or tissue may be monitored, for example, for ion transport/exchange activity, cell division, and/or cell viability. In some aspects, it may be useful to simulate an active load on an organ or portion thereof during recellularization. In some aspects, a computer-readable storage medium in combination with a programmable processor, may be used to coordinate the components used to monitor and maintain an active load on an organ or tissue. In some cases, the weight of an organ or portion thereof or tissue may be entered into a computer-readable storage medium as described herein, which, in combination with a programmable processor, can calculate exposure times and perfusion pressures for that particular organ or tissue. Such a storage medium may record preload and afterload (the pressure before and after perfusion, respectively) and the rate of flow. In this embodiment, for example, a computer-readable storage medium in combination with a programmable processor can adjust the perfusion pressure, the direction of perfusion, and/or the type of perfusion solution via one or more pumps and/or valve controls.

In some aspects, immersion-based decellularization of an organ or portion thereof can be performed. In some aspects, whole organs or portions thereof can be decellularized by removing the entire cellular and tissue content from the organ. In some aspects, decellularization can comprise a series of sequential extractions. In some aspects, a first step can involve removal of cellular debris and solubilization of a cell membrane. This can be followed by solubilization of the nuclear cytoplasmic components and the nuclear components. In some aspects, an organ can be decellularized by removing the cell membrane and cellular debris surrounding the organ using gentle mechanical disruption methods. The gentle mechanical disruption methods can disrupt the cellular membrane. However, the process of decellularization should avoid damage or disturbance of the biostructure's complex infra-structure. Gentle mechanical disruption methods can include scraping the surface of the organ, agitating the organ, or stirring the organ in a suitable volume of fluid, e.g., distilled water. In some aspects, the gentle mechanical disruption method can include magnetically stirring (e.g., using a magnetic stir bar and a magnetic plate) the organ or portion thereof in a suitable volume of distilled water until the cell membrane is disrupted and the cellular debris has been removed from the organ or portion thereof. After the cell membrane has been removed, the nuclear and cytoplasmic components of the biostructure are removed. This can be performed by solubilizing the cellular and nuclear components without disrupting the infra-structure. To solubilize the nuclear components, non-ionic detergents or surfactants may be used. Examples of nonionic detergents or surfactants include, but are not limited to, the Triton series, available from Rohm and Haas of Philadelphia, Pa., which includes Triton X-100, Triton N-101, Triton X-114, Triton X-405, Triton X-705, and Triton DF-16, available commercially from many vendors; the Tween series, such as monolaurate (Tween 20), monopalmitate (Tween 40), monooleate (Tween 80), and polyoxethylene-23-lauryl ether (Brij. 35), polyoxyethylene ether W-1 (Polyox), and the like, sodium cholate, deoxycholates, CHAPS, saponin, n-Decyl β-D-glucopuranoside, n-heptyl β-D glucopyranoside, n-Octylα-D-glucopyranoside and Nonidet P-40.

In some cases, physical treatment of an organ or portion thereof can be done to achieve decellularization. Physical treatment can be used to lyse, kill, and remove cells from an ECM or portion thereof. Physical treatment can utilize temperature, force, pressure, and electrical disruption. In some cases, temperature methods can be used in a rapid freeze-thaw mechanism. For example, by freezing a tissue, microscopic ice crystals can form around the plasma membrane and the cell can be lysed. After lysing the cells, the tissue can be further exposed to liquidized chemicals that can degrade and wash out any residual or undesirable components. In some cases, temperature methods can conserve the physical structure of the ECM scaffold. An organ or portion thereof, and a tissue can be decellularized at a suitable temperature. A suitable temperature can be from about 4° C., 8° C., 10° C., 12° C., 14° C., 16° C., 18° C., 20° C., 22° C., 24° C., 26° C., 28° C., 30° C., 32° C., 34° C., 36° C., 38° C., 40° C., 45° C., 50° C., 55° C., 60° C., or up to about 70° C. A physical treatment can also include the use of pressure. Pressure decellularization can involve the controlled use of hydrostatic pressure applied to a tissue, organ, or portion thereof. Pressure decellularization can be performed at high temperatures in some cases to avoid unmonitored ice crystal formation. In some cases, Electrical disruption of an organ or portion thereof can be performed. Electrical disruption can be done to lyse cells housed in a tissue or organ. By exposing a tissue, organ, or portion thereof to electrical pulses, micropores can be formed at the plasma membrane. The cells can die after their homeostatic electrical balance is ruined through the applied stimulus. This electrical process is documented as Non-thermal irreversible electroporation (NTIRE).

In some cases, chemical treatment of an organ or portion thereof can be performed to achieve decellularization. Chemicals and/or salts thereof for use in a chemical treatment can be selected for decellularization depending on the thickness, extracellular matrix composition, and intended use of the tissue or organ. For example, enzymes may not be used on a collagenous tissue because they disrupt the connective tissue fibers. However, when collagen is not present in a high concentration or needed in the tissue, enzymes can be a viable option for decellularization. The chemicals and/or salts thereof can be used to kill and remove cells can be but are not limited to acids, alkaline treatments, ionic detergents, non-ionic detergents, and zwitterionic detergents. In some cases, one or more chemicals can comprise a cellular disruption media. A cellular disruption medium can comprise at least one detergent such as Sodium dodecyl sulfate (SDS), polyethylene glycol (PEG), or Triton X. Detergents can act effectively to lyse the cell membrane and expose the contents to further degradation. For example, after SDS lyses a cellular membrane, endonucleases and/or exonucleases can degrade the genetic contents, while other components of the cell can be solubilized and washed out of the matrix. In some cases, a detergent can be mixed with an alkaline and/or acid treatments due to their ability to degrade nucleic acids and solubilize cytoplasmic inclusions.

One or more cellular disruption media can be used to decellularize an organ or tissue. A cellular disruption medium can comprise at least one detergent such as SDS, PEG, or Triton X

A detergent can be administered for more than, less than or equal to about: 10 min, 30 min, 60 min, 1 hr., 2 hrs., 3 hrs., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some cases, a detergent can be contacted with the organ or portion thereof for more than, less than or equal to about: 2 hours, 3 hours, 4 hours, 5 hours, 6 hours, 7 hours, 8 hours, 9 hours, 10 hours, 11 hours, 12 hours, 13 hours, 14 hours, 15 hours, or about 20 hours per gram of solid organ or tissue with cellular disruption medium.

Including washes, an organ may be perfused for up to about 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some cases, an organ or portion thereof can be perfused from about 12 hours to about 72 hours per gram of tissue. In some aspects, perfusion can be adjusted to physiologic conditions including pulsatile flow, rate, pressure, and any combination thereof.

In some cases, a sequential method of decellularization can comprise contacting the organ or portion thereof with a cellular disruption media, such as an SDS detergent, followed by a washing step, followed by the addition of one or more chemicals, followed by contacting with a detergent, and ending with at least one wash step. A sequential method of decellularization can comprise at least 1, 2, 3, 4, 5, 6, 7, 8, 9, 10, 11, 12, 13, 14, or up to 15 contacting steps with any media or solution provided herein.

A buffer provided herein can be at a concentration (volume/volume or weight to weight) of more than, less than or equal to about: 0.1%, 0.5%, 1%, 1.5%, 2%, 2.5%, 3%, 3.5%, 4%, 4.5%, 5%, 10%, 20%, 30%, 40%, 50%, 60%, 70%, 80%, 90%, 95%, or 99%. In some cases, a buffer provided herein can be at a concentration of about 100%.

### Methods of Engrafting Cells

Disclosed herein are methods for engrafting cells such as engineered podocyte-like cells on decellularized organs or portions thereof. In some cases, HUVEC cells can be engrafted on a decellularized organ or a portion thereof with or without engineered podocyte-like cells.

In some cases, decellularization removes the cellular material from a kidney, such as a porcine kidney to generate a three-dimensional, human-scale-scaffold composed of an extracellular matrix that maintains the complex architecture of the native kidney. This decellularized extracellular matrix can then be repopulated with human cells, such as engineered podocyte-like cells derived from appropriate human kidney donors to produce a functional bioengineered kidney graft. In some instances, the decellularized extracellular matrix is repopulated with HUVEC cells. The methods herein describe recellularization of an extracellular matrix.

Such methods include methods of engrafting cells on an at least partially decellularized kidney extracellular matrix comprising: contacting the at least partially decellularized kidney extracellular matrix with a plurality of engineered podocyte-like cells and/or a plurality of HUVEC cells. In some cases, the contacting occurs in a bioreactor chamber. In some cases, the contacting comprises depositing through a ureter of the at least partially decellularized kidney extracellular matrix the plurality of the engineered podocyte-like cells in an aqueous composition into a glomerulus of the at least partially decellularized kidney extracellular matrix, thereby engrafting cells on the at least partially decellularized kidney extracellular matrix. In some cases, depositing through the ureter comprises creating a vacuum in the bioreactor chamber.

In some cases, a method can further comprise seeding one or more additional cell types. In some cases, the method can comprise seeding a plurality of engineered podocyte-like cells. In some cases, the method can further comprise seeding a plurality of mesangial cells, a plurality of human umbilical vein endothelial cells (HUVEC), or both. In some cases, the method can further comprise seeding a plurality of tubule epithelial cells, macula densa cells, glomerular endothelial cells, a tubule cell, a podocyte, a smooth muscle cell, a pericyte, a juxtaglomerular cell, collecting duct cells (e.g., CD-PC, CD-Trans, or CD-IC), a distal convoluted tubule cells (e.g., DCT1, DCT2), a loop of Henle cell, a proximal tubule cell (e.g., convoluted or straight), vas afferens cells, vas efferens cells, peritubular capillary cells, ascending vasa recta cells, descending vasa recta cells immune cells, mesangial cells, parietal epithelial cells or any combination thereof. In some cases, the method can comprise seeding an endothelial cell, a human umbilical vein endothelial cell (HUVEC), or both. In some cases, a cell can be cryopreserved prior to seeding. A cell can be any animal cell, for example a human cell, a pig cell, a sheep cell, a goat cell, a monkey cell, a cow cell, a dog cell, a cat cell, or a mixture thereof. In some cases, a cell can be an autologous cell, a xenogeneic cell, or an allogeneic cell to the decellularized organ.

In some embodiments, after the engrafting the cells are grown in the extracellular matrix. In some cases, media is continuously perfused through the recellularized kidney after the grafting to provide nutrients for the engrafted cells. In some cases, the media is replaced with new media after more than, less than, or equal to about: 1, 6, 12, 24, 48, 72, or 96 hours of cell growth.

Provided herein are also compositions and methods of generating engineered organs or portions thereof comprising a population of cells. In some aspects, at least two populations of cells can be introduced into a decellularized organ or portion thereof. In some cases, an least partially recellularized isolated organ or portion thereof comprises a kidney or a portion thereof.

Decellularized organs and portions thereof provided herein can be recellularized. An organ or tissue can be generated by contacting a decellularized organ or tissue as described herein with a population of cells. In some aspects, a population of cells can comprise an engineered podocyte-like cell. In some cases, a population of cells can be undifferentiated cells, partially differentiated cells, or fully differentiated cells. In some cases, the number of cells that can be introduced into a decellularized organ or portion thereof in order to generate an organ or tissue can be dependent on both the organ (e.g., which organ, the size and weight of the organ) or tissue and the type and developmental stage the cells. Different types of cells may have different tendencies as to the population density those cells will reach. Similarly, different organ or tissues may be recellularized at different densities. By way of example, a decellularized organ or tissue can be “seeded” with more than, less than, or equal to about: 100, 1,000 10,000, 100,000, 1,000,000, 10,000,000, or 100,000,000) cells (e.g., engineered podocyte like cells); or can have from about 1,000 cells/mg tissue (wet weight) to about 10,000,000 cells/mg tissue (wet weight) attached thereto. In some aspects, cells can be introduced (“seeded”) into a decellularized organ or tissue by injection, physical placement, and/or depositing into one or more locations.

Cells herein can be further cultured under conditions that result in fully differentiated cells. Additionally, or alternatively, cells can be obtained from any number of sources such as blood, kidney, any other tissue or organ that harbors cells. For example, representative cells can comprise tubule epithelial cells, macula densa cells, glomerular endothelial cells, podocytes, a smooth muscle cell, a pericyte, a juxtaglomerular cell, collecting duct cells (e.g., CD-PC, CD-Trans, or CD-IC), a distal convoluted tubule cells (e.g., DCT1, DCT2), a loop of Henle cell, a proximal tubule cell (e.g., convoluted or straight), vas afferens cells, vas efferens cells, peritubular capillary cells, ascending vasa recta cells, descending vasa recta cells immune cells, mesangial cells, parietal epithelial cells or any combination thereof. A cell can be any animal cell, for example a human cell, a pig cell, a sheep cell, a goat cell, a monkey cell, a cow cell, a dog cell, a cat cell, or a mixture thereof. In some cases, a cell can be an autologous cell, a xenogeneic cell, or an allogeneic cell. In some cases, a cell can be a stem cell, such as embryonic stem cells, umbilical cord blood cells, tissue-derived stem or progenitor cells, bone marrow-derived stem or progenitor cells, blood-derived stem or progenitor cells, adipose tissue-derived stem or progenitor cells, mesenchymal stem cells (MSC), skeletal muscle-derived cells, induced pluripotent stem cells (iPSCs), genetically modified cells removing immunogenic factors including but not limited to HLA, or multipotent adult progenitor cells.

A composition that includes cells herein can be delivered to a tissue or organ matrix in a solution that is compatible with the cells (e.g., in a physiological composition) under physiological conditions (e.g., 37° C.) and under non-physiologic conditions (e.g. 4-35° C.). A physiological composition, as referred to herein, can include, without limitation, buffers, nutrients (e.g., sugars, carbohydrates), enzymes, expansion and/or differentiation medium, cytokines, antibodies, repressors, growth factors, salt solutions, or serum-derived proteins.

In some cases, cells can be introduced into an organ or tissue matrix by perfusion. Perfusion can occur via the vasculature or vasculature-type structure of the organ or tissue matrix. Perfusion to recellularize an organ or tissue matrix can be at a flow rate that is sufficient to circulate the physiological composition of cells through the vasculature. Perfusion with cells can be multi-directional (e.g., antegrade and retrograde). Perfusion of cells may be followed by a static hold time to enhance engraftment prior to reperfusion of the organ or tissue matrix.

In some aspects, at least one type of cell can be introduced into a decellularized organ or portion thereof. For example, a cocktail of cells or a population of cells can be injected and/or deposited at multiple positions in a decellularized organ or tissue or different cell types can be injected and/or deposited into different portions of a decellularized organ or portion thereof. Alternatively, or in addition to injection, cells or a cocktail of cells can be introduced by perfusion into a cannulated decellularized organ or portion thereof. For example, cells can be perfused into a decellularized organ using a perfusion medium, which can then be changed to an expansion and/or differentiation medium to induce growth and/or differentiation of the cells. During recellularization, an organ or tissue can be maintained under conditions in which at least some of the cells can proliferate, multiply, differentiate, and any combination thereof in the decellularized organ or portion thereof. In some aspects, those conditions can include, without limitation, the appropriate temperature, pressure, electrical activity, mechanical activity, force, the appropriate amounts of O2 and/or CO2, an appropriate amount of humidity, sterile or near-sterile conditions, and any combination thereof. During recellularization, the decellularized organ or tissue and the cells attached thereto can be maintained in a suitable environment. For example, the engineered podocyte-like cells may require a nutritional supplement (e.g., nutrients and/or a carbon source such as glucose), exogenous hormones or growth factors, and/or a particular pH. In some embodiments, cells as provided herein can be allogeneic, xenogeneic, or autologous to a decellularized organ or portion thereof.

In some embodiments, an engineered podocyte-like cell can be deposited into a decellularized kidney. Populations of cells may engraft onto the decellularized kidney matrix. In some cases, once engrafted onto the kidney matrix the engineered podocyte-like cells may have increased gene expression in NPHS1, NPHS2, and/or SYNPO. In some cases, once engrafted onto the kidney matrix the engineered podocyte-like cells may have increased protein expression of podocin, nephrin, podocalyxin and/or synaptopodin.

### Recellularized Organs

Disclosed herein are recellularized organs or portions thereof such as recellularized kidneys. In some cases, a recellularized organ has been recellularized with engineered podocyte-like cells. A recellularized organ herein can have a similar function to a wild-type or a primary organ such that the organ is able to sustain life in an animal. In some cases, a recellularized organ scaffold (e.g., a decellularized organ or portion thereof) is xenogeneic, allogeneic, or autologous to one or more cell populations used to recellularized the decellularized organ or portion thereof. In some cases, the one or more cell populations can be xenogeneic, allogeneic, or autologous to a decellularized organ or portion thereof.

For example, an at least partially recellularized organ or a portion thereof can comprise the engineered podocyte-like cell described herein. In some cases, the least partially recellularized isolated organ or portion thereof in a closed loop normothermic perfusion system sustains urine/serum protein values in urine of less than or equal to 30% at about 1 hour post normothermic perfusion and/or less than or equal to 65% at about 4 hours post implantation. In some cases, the percentage values reflect a urine/serum normalized value. The normalized values can be calculated as ([urine total protein (g/L)]/[serum total protein (g/L)])*100. The percentages of urine/serum protein levels in urine are used to show the concentration of protein in the urine as compared to the concentration of protein in the perfusing blood. For example, a urine/serum normalized protein of less than 1% shows that the concentration of protein in the urine is less than 1% of that perfusing in the blood serum. In some cases, the urine protein levels are sustained in a liquid. In some cases, the least partially recellularized isolated organ or portion thereof in a closed loop normothermic perfusion system sustains urine/serum protein values in urine of less than, more than, or equal to about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% at about 1, 2, 3, 4, or 5 hour(s) post normothermic perfusion. In some cases, the least partially recellularized isolated organ or portion thereof in a closed loop normothermic perfusion system sustains urine/serum protein values in urine of about: 1% to about 40%, 1% to about 30%, 10% to about 35%, 15% to about 30%, 15% to about 25%, 20% to about 30%, or 25% to about 35% at about 1, 2, 3, 4, or 5 hour(s) post normothermic perfusion. In some cases, the least partially recellularized isolated organ or portion thereof post implantation sustains urine/serum protein values in urine of less than, more than, or equal to about: 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% at about 1, 2, 3, 4, or 5 hour(s) post implantation. In some cases, the least partially recellularized isolated organ or portion thereof post implantation sustains urine/serum protein values in urine of about: 20% to about 80%, 30% to about 70%, 35% to about 65%, 45% to about 70%, 50% to about 60%, 30% to about 55%, or 55% to about 65% at about 1, 2, 3, 4, or 5 hour(s) post implantation.

In some cases, the least partially recellularized isolated organ or portion thereof post implantation sustains urine protein values of less than, more than, or equal to about: 5 g/L (gram/liter), 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, or 45 g/L at about 1, 2, 3, 4, or 5 hour(s) post implantation. In some cases, the least partially recellularized isolated organ or portion thereof post implantation sustains urine protein values of about: 5 g/L to about 45 g/L, 10 g/L to about 40 g/L, 20 g/L to about 35 g/L, 25 g/L to about 35 g/L, 20 g/L to about 30 g/L, 24 g/L to about 32 g/L, or 30 g/L to about 40 g/L at about at about 1, 2, 3, 4, or 5 hour(s) post implantation. In some cases, the least partially recellularized isolated organ or portion thereof in a closed loop normothermic perfusion system sustains urine protein values of less than, more than, or equal to about: 0.01 g/L (grams/liter), 0.05 g/L, 0.1 g/L, 0.15 g/L, 0.2 g/L, 0.3 g/L, 0.4 g/L, 0.5 g/L, 0.6 g/L, 0.7 g/L, 0.8 g/L, 0.9 g/L, 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, or 20 g/L at about 30 min or 1 hour post normothermic perfusion. In some cases, the least partially recellularized isolated organ or portion thereof in a closed loop normothermic perfusion system sustains urine protein values of about: 0.1 g/L to about 20 g/L, 0.1 g/L to about 1 g/L, 1 g/L to about 10 g/L, 5 g/L to about 15 g/L, 8 g/L to about 12 g/L, 5 g/L to about 10 g/L, or 10 g/L to about 17 g/L at about 30 min or 1 hour post normothermic perfusion. In some cases, normal urine protein is about 0.15 g/L with a primary kidney. In some cases, normal urine protein is about 10 g/L with an least partially recellularized isolated organ or portion thereof. In some cases, a urine protein value can be determined from the amount of protein in a liquid before and after circulation through the recellularized organ or a portion thereof.

In some cases, the least partially recellularized isolated organ or portion thereof in a closed loop normothermic perfusion system sustains serum protein values of less than, more than, or equal to about: 40 g/L (grams/liter), 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, or 75 g/L at about 30 min or 1 hour post normothermic perfusion. In some cases, the least partially recellularized isolated organ or portion thereof in a closed loop normothermic perfusion system sustains serum protein values of about: 40 g/L to about 75 g/L, 50 g/L to about 70 g/L, 55 g/L to about 65 g/L, 60 g/L to about 70 g/L, 65 g/L to about 75 g/L, 58 g/L to about 65 g/L, or 63 g/L to about 68 g/L at about 30 min or 1 hour post normothermic perfusion.

In some embodiments, the least partially recellularized isolated organ or portion thereof in a closed loop normothermic perfusion system sustains urine hematocrit levels of less than or equal to 30% at 1 hour post normothermic perfusion, and/or less than or equal to 1% at 4 hours post implantation. In some cases, these percentage values reflect a urine/serum normalized value. The normalized values can be calculated as ([urine hematocrit %]/[serum hematocrit %])*100. The percentages of urine/serum hematocrit levels are used to show the concentration of red blood cells in the urine as compared to of the concentration of red blood cells in the perfusing blood. In some cases, the urine/serum hematocrit levels are sustained in a liquid. In some cases, the least partially recellularized isolated organ or portion thereof in a closed loop normothermic perfusion system sustains urine/serum hematocrit levels in urine of less than, more than, or equal to about: 0.01%, 0.02%, 0.03%, 0.04%, 0.05%, 0.06%, 0.07%, 0.08%, 0.09%, 0.1%, 0.1%, 0.2%, 0.3%, 0.4%, 0.5%, 0.6%, 0.7%, 0.8%, 0.9%, 1%, 0.5, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% at about 30 min, 1 hour, 2 hours, 3 hours, 4 hours, or 5 hours post normothermic perfusion. In some cases, the least partially recellularized isolated organ or portion thereof in a closed loop normothermic perfusion system sustains urine/serum hematocrit levels in urine of about: 1% to about 40%, 1% to about 30%, 10% to about 35%, 15% to about 30%, 15% to about 25%, 20% to about 30%, or 25% to about 35% at about 30 min, 1 hour, 2 hours, 3 hours, 4 hours, or 5 hours post normothermic perfusion. In some cases, a urine hematocrit level can be determined from the amount of hematocrit in a liquid before and after circulation through the recellularized organ or a portion thereof.

In some cases, the least partially recellularized isolated organ or portion thereof in a closed loop normothermic perfusion system sustains urine/serum hematocrit levels in serum of less than, more than, or equal to about: 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%, at about 30 min, 1 hour, 2 hours, 3 hours, 4 hours, or 5 hours post normothermic perfusion. In some cases, the least partially recellularized isolated organ or portion thereof in a closed loop normothermic perfusion system sustains urine/serum hematocrit levels in serum of about: 25% to about 55%, 25% to about 45%, 30% to about 45%, 35% to about 45%, 40% to about 45%, 40% to about 50%, or 45% to about 55% at about 30 min, 1 hour, 2 hours, 3 hours, 4 hours, or 5 hours post normothermic perfusion.

In some cases, the least partially recellularized isolated organ or portion thereof post implantation sustains urine/serum hematocrit levels in urine of less than, more than, or equal to about: 0.01%, 0.02%, 0.03%, 0.04%, 0.05%, 0.06%, 0.07%, 0.08%, 0.09%, 0.1%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% at about 1, 2, 3, 4, or 5 hour(s) post implantation. In some cases, the least partially recellularized isolated organ or portion thereof post implantation sustains urine/serum hematocrit levels in urine of about: 0.01% to about 20%, 0.1% to about 3%, 0.1% to about 1%, 0.5% to about 1.5%, 0.5% to about 4%, 1% to about 5%, 3% to about 10%, or 8% to about 18% at about 1, 2, 3, 4, or 5 hour(s) post implantation. For example, the percentages of urine/serum hematocrit levels are used to show the concentration of red blood cells in the urine as compared to of the concentration of red blood cells in the perfusing blood. So a urine/serum hemocrit value of less than 1% shows the concentration of red blood cells in the urine is less than 1% of the concentration of red blood cells in the perfusing blood.

In some embodiments, the least partially recellularized isolated organ or portion thereof post implantation can sustain a urine flow rate of less than, more than, or equal to about: 1 mL/h (hour), 2 mL/h, 3 mL/h, 4 mL/h, 5 mL/h, 6 mL/h, 7 mL/h, 8 mL/h, 9 mL/h, 10 mL/h, 11 mL/h, 12 mL/h, 13 mL/h, 14 mL/h, 15 mL/h, 16 mL/h, 17 mL/h, 18 mL/h, 19 mL/h, 20 mL/h, 21 mL/h, 22 mL/h, 23 mL/h, 24 mL/h, 25 mL/h, 26 mL/h, 27 mL/h, 28 mL/h, 29 mL/h, 30 mL/h, 31 mL/h, 32 mL/h, 33 mL/h, 34 mL/h, or 35 mL/h. In some embodiments, the least partially recellularized isolated organ or portion thereof post implantation can sustain a urine flow rate of about: 1 mL/h to about 35 mL/h, 10 mL/h to about 25 mL/h, 5 mL/h to about 15 mL/h, 10 mL/h to about 20 mL/h, 15 mL/h to about 25 mL/h, 18 mL/h to about 24 mL/h, or 20 mL/h to about 30 mL/h. In some cases, urine flow rate can be determined after 10 min, 20 min, 30 min, 40 min, 50 min, 60 min, 1.5 hours, 2 hours, 3 hours, 4 hours, 5 hours, or more than 5 hours post implantation.

In some embodiments, the least partially recellularized isolated organ or portion thereof in a closed loop normothermic perfusion system can sustain a urine flow rate of less than, more than, or equal to about: 1 mL/h (hour), 2 mL/h, 3 mL/h, 4 mL/h, 5 mL/h, 6 mL/h, 7 mL/h, 8 mL/h, 9 mL/h, 10 mL/h, 11 mL/h, 12 mL/h, 13 mL/h, 14 mL/h, 15 mL/h, 16 mL/h, 17 mL/h, 18 mL/h, 19 mL/h, 20 mL/h, 21 mL/h, 22 mL/h, 23 mL/h, 24 mL/h, 25 mL/h, 26 mL/h, 27 mL/h, 28 mL/h, 29 mL/h, 30 mL/h, 31 mL/h, 32 mL/h, 33 mL/h, 34 mL/h, or 35 mL/h. In some embodiments, the least partially recellularized isolated organ or portion thereof in a closed loop normothermic perfusion system can sustain a urine flow rate of about: 1 mL/h to about 35 mL/h, 10 mL/h to about 25 mL/h, 5 mL/h to about 15 mL/h, 10 mL/h to about 20 mL/h, 15 mL/h to about 25 mL/h, 18 mL/h to about 24 mL/h, or 20 mL/h to about 30 mL/h. In some cases, urine flow rate can be determined after 10 min, 20 min, 30 min, 40 min, 50 min, 60 min, 1.5 hours, 2 hours, 3 hours, 4 hours, 5 hours, or more than 5 hours in a closed loop normothermic perfusion system.

In some embodiments, the least partially recellularized isolated organ or portion thereof in a closed loop normothermic perfusion system or post implantation can sustain a flow rate of less than, more than, or equal to about: 0.1 L/min (minute), 0.2 L/min, 0.3 L/min, 0.4 L/min, 0.5 L/min, 0.6 L/min, 0.7 L/min, 0.8 L/min, 0.9 L/min, 1 L/min, 2 L/min, 3 L/min, 4 L/min, or 5 L/min after about at about 1, 2, 3, 4, or 5 hour(s). In some embodiments, the least partially recellularized isolated organ or portion thereof in a closed loop normothermic perfusion system or post implantation can sustain a flow rate of about: 0.1 L/min (minute) to about 5 L/min, 0.1 L/min to about 1.5 L/min, 0.5 L/min to about 2 L/min, or 1 L/min to about 3 L/min.

In some embodiments, the least partially recellularized isolated organ or portion thereof in a closed loop normothermic perfusion system or post implantation can sustain a packed cell volume (PCV) as determined by normalized values (e.g., ([urine value]/[serum value])*100) in urine of more than, or equal to about: 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%. In some embodiments, the least partially recellularized isolated organ or portion thereof in a closed loop normothermic perfusion system or post implantation can sustain a packed cell volume (PCV) (%) as determined by normalized values (e.g., ([urine value]/[serum value])*100) in urine of about: 0.1% to about 35%, 1% to about 10%, 0.5% to about 5%, 5% to about 20%, 10% to about 15%, 25% to about 35%, 20% to about 30%, 10% to about 35%, or 30% to about 40%.

In some embodiments, the closed loop normothermic perfusion system can determine levels of biological compounds such as metabolites in a circulating fluid. In some cases, the closed loop normothermic perfusion system can determines levels of a creatinine, a urea, a sodium, a potassium, a glucose, a lactate, a bicarbonate, a salt, a blood component, a protein, or any combination thereof. In some cases, a sample can be acquired from the closed loop normothermic perfusion system before and after circulation in a recellularized organ or portion thereof to determine the levels of a compound.

### Uses of Recellularized Organs

Disclosed herein are methods of use of recellularized organs or portions thereof. In some cases a method herein can comprise implanting recellularized organ or portions thereof to a subject in need thereof, for example a subject with a kidney disease. In some cases, a method of treating a disease can comprise implanting a recellularized organ or a portion thereof. Recellularized and recellularized organs or portions thereof provided herein can be used in a variety of applications. For example, organs or portions thereof can be implanted into a subject. In some aspects, a composition herein, such as an organ or portion thereof, may be transplanted into a subject that has a disease. Relevant diseases that may require organ transplantation include but are not limited to: organ failure, cardiomyopathy, cirrhosis, chronic obstructive pulmonary disease, pulmonary edema, biliary atresia, emphysema and pulmonary hypertension, coronary heart disease, valvular heart disease, congenital heart disease, coronary artery disease, pancreatitis, cystic fibrosis, diabetes, hepatitis, hypertension, idiopathic pulmonary fibrosis, polycystic kidneys, short gut syndrome, injury, birth defects, genetic diseases, autoimmune disease, and any combination thereof. In some cases, a disease can comprise a kidney disease. In some cases, a disease can comprise end-stage renal disease. In some cases, a kidney disease can comprise a Fabry disease, a cystinosis, a glomerulonephritis, an IgA nephropathy, a lupus nephritis, an atypical hemolytic uremic syndrome, a polycystic kidney disease. In some cases, a kidney disease can comprise a chronic kidney disease, or an acute kidney disease. In some cases a disease can comprise an acute kidney injury. In some cases, a disease can comprise Alport syndrome, amyloidosis, Goodpasture's disease, a glomerular disease, an infection disease, an interstitial nephritis, a Lupus nephritis, a nephrotic syndrome, a renal tubular acidosis, a solitary kidney or any combination thereof. In some cases, implants can be used to replace or augment existing tissue. For example, to treat a subject with a kidney disorder by replacing the dysfunctional kidney of the subject with an exogenous or engineered kidney, such as the recellularized kidney described herein. The subject can be monitored after implantation of the exogenous kidney, for amelioration of the kidney disorder. Any decellularized organ or portion thereof provided herein can be utilized for implantation into a subject.

In some cases, a composition provided herein, such as a solid organ or portion thereof can have from about 1% to about 100% of its native function after decellularization. In some cases, a composition provided herein, such as a solid organ or portion thereof can have from about 5%, 10%, 20%, 30%, 40%, 50%, 60%, 70%, 80%, 90%, 95%, or up to about 100% of its native function after decellularization. In some cases, a composition provided herein, such as a recellularized organ or a portion thereof can have from about 1% to about 100% of its native function after recellularization. In some cases, a composition provided herein, such as a recellularized organ or a portion thereof can have more than, less than, or equal to about: 5%, 10%, 20%, 30%, 40%, 50%, 60%, 70%, 80%, 90%, 95%, or up to about 100% of its native function after recellularization.

In some aspects, particular organs or portions thereof may be suitable for transplantation when they function below that of their native counterpart. For example, a kidney may need approximately from about 20% of the total organ function to provide the needed organ function to save a person from kidney failure. In some aspects, a kidney may need approximately from about 20-30%, 30-40%, 20-50%, 20-60%, 40-60% of the total organ function to be suitable for transplantation. In some aspects, an organ may function equal to a native counterpart.

In some cases, a lifespan of a subject may be extended after transplantation of a composition, such as an organ or portion thereof provided herein. For example, a lifespan of a subject may be extended from about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, or up to about 100 years after transplantation. In some aspects, transplantation of a composition, such as a recellularized organ or a portion thereof provided herein, may reduce the need of a secondary treatment in a subject. Secondary treatments can refer to dialysis (e.g., hemodialysis and/or peritoneal dialysis), pacemakers, respirators, and combinations thereof. In some cases, a secondary treatment can be a medication such as a kidney mediation. In some cases, a method herein can comprise further administering a secondary treatment to a subject who receives a transplantation.

Decellularized and recellularized organs or portions thereof provided herein can also be used in vitro to screen a wide variety of compounds, for effectiveness and cytotoxicity of pharmaceutical agents, chemical agents, growth/regulatory factors. The cultures can be maintained in vitro and exposed to the compound to be tested. The activity of a cytotoxic compound can be measured by its ability to damage or kill cells in culture. This may readily be assessed by staining techniques. The effect of growth/regulatory factors may be assessed by analyzing the cellular content of the matrix, e.g., by total cell counts, and differential cell counts. This may be accomplished using standard cytological and/or histological techniques including the use of immunocytochemical techniques employing antibodies that define type-specific cellular antigens. The effect of various drugs on normal cells cultured in the reconstructed artificial organs may be assessed.

Decellularized and recellularized organs or portions thereof provided herein can be used in vitro to filter aqueous solutions, for example, an engineered artificial kidney may be used to filter blood. Using the engineered kidney provides a system with morphological features that resemble the in vivo kidney products. This system may be suitable for hemodialysis. In some aspects, the system may also be useful for hemofiltration to remove water and low molecular weight solutes from blood. The artificial kidney may be maintained in vitro and exposed to blood which may be infused into the luminal side of the artificial kidney. The processed aqueous solution may be collected from the abluminal side of the engineered kidney. The efficiency of filtration may be assessed by measuring the ion, or metabolic waste content of the filtered and unfiltered blood.

Decellularized and recellularized organs or portions thereof provided herein can be used as a vehicle for introducing genes and gene products in vivo to assist or improve the results of the transplantation and/or for use in gene therapies. For example, cultured cells, such as kidney cells, can be engineered to express gene products. The cells can be engineered to express gene products transiently and/or under inducible control or as a chimeric fusion protein anchored to the cells. In another embodiment, the cells can be genetically engineered to express a gene for which a patient is deficient, or which would exert a therapeutic effect. The genes of interest engineered into the cells may be related to the disease being treated. For example, for a kidney disorder, the endothelial or cultured kidney cells can be engineered to express gene products that would ameliorate the kidney disorder.

Provided herein are also compositions and methods of generating engineered organs or portions thereof comprising a population of cells, such as engineered podocyte-like cells. In some aspects, at least two populations of cells can be introduced into a decellularized organ or portion thereof. Organs that can be engineered include, but are not limited to, heart, kidney, liver, pancreas, spleen, urinary bladder, ureter, urethra, skeletal muscle, small and large bowel, esophagus, stomach, brain, spinal cord and bone. In some case an organ that can be engineered includes a kidney.

In some cases, a recellularized kidney can be transplanted into a recipient. A recellularized kidney as described herein can be transplanted as a functional organ. In some cases, function can be determined through filtration of a liquid by the recellularized organ. In some embodiments, functionality can be assessed by determining consumption of certain metabolites (i.e. glucose, lactate, glutamine, glutamate and ammonia). Such consumption can be determined by perfusing in a continuous line of the metabolite and measuring a rate of consumption of the metabolite over time using, for example, a change in electrochemical potential. In some cases, the rate of consumption of a metabolite can be used to determine successful engraftment of endothelial cells onto a decellularized matrix.

In some cases, a recellularized kidney can be transplanted along with systemic administration of an immunosuppressor. Administration of an immunosuppressor may prolong patency of a transplanted organ. In some cases, an immunosuppressor can be a corticosteroid, a Janus kinase inhibitor, a calcineurin inhibitor, an mTOR inhibitor, an IMDH inhibitor, a biologic, a monoclonal antibody, or any combination thereof. Examples of corticosteroids can include prednisone, budesonide, prednisolone, and methylprednisolone. Examples of Janus kinase inhibitors can include tofacitinib. Examples of calcineurin inhibitors can include cyclosporine and tacrolimus. Examples of mTOR inhibitors can include sirolimus and everolimus. Examples of IMDH inhibitors can include azathioprine, leflunomide, and mycophenolate. Examples of immunosuppressive biologics can include abatacept, adalimumab, anakinra, certolizumab, etanercept, golimumab, infliximab, ixekizumab, natalizumab, rituximab, secukinumab, tocilizumab, ustekinumab, and vedolizumab. Examples of immunosuppressive monoclonal antibodies can include basiliximab and daclizumab. Such immunosuppressors can be administered to a recipient of a recellularized kidney via enteral routes (including oral, gastric or duodenal feeding tube, rectal suppository and rectal enema), parenteral routes (injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal or topical (including epicutaneous, dermal, enema, eye drops, ear drops, intranasal, vaginal) administration. Immunosuppressors can be administered to a recipient at a dose of from about: 0.001 mg to about 1 mg, 0.01 mg to about 1 mg, 0.1 mg to about 10 mg, 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

### Kits

Also disclosed herein are kits. In some cases, a kit can comprise a composition described herein. In some embodiments, a kit can comprise one or more medias disclosed herein, such as a first media and/or a second media in a container. In some cases, a kit can comprise a cell such as a glomerular cell or an engineered podocyte-like cell. In some cases, a kit can comprise an at least partially recellularized organ or a portion thereof and a container. In some cases, a kit can comprise an at least partially decellularized organ or a portion thereof and a container. A kit herein can comprise a container. In some cases, a container can comprise glass, metal, plastic, and/or suitable material for a container.

In some embodiments, a kit can comprise a first media for culturing podocyte-like cells comprising at least one of: a retinoic acid, a salt thereof, a corticosteroid, a salt thereof, a calcitriol, or a salt thereof in a container, and/or a second media for culturing podocyte-like cells comprising at least one of: a SB431542, a salt thereof, an IWR-1-endo, or a salt thereof in a container. In some cases, a first media, a second media, or both can comprise a glomerular cell.

### EXAMPLES

### Example 1: Making Engineered Podocyte-Like Cells

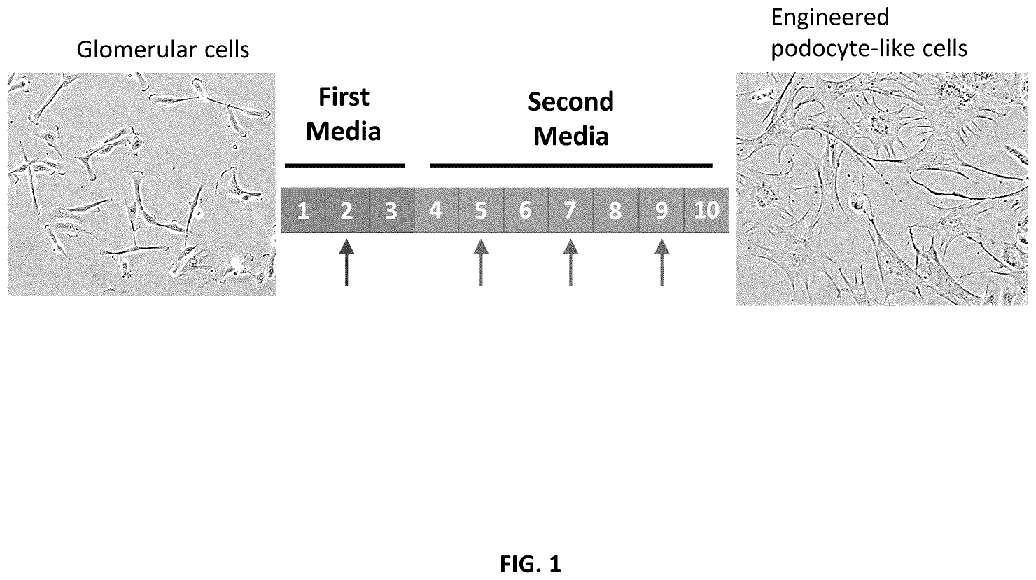

To initiate the podocyte differentiation protocol, glomerular outgrowth cells are cultured in a first media for a total of 3 days with a first media change occurring 48 hours after the initial culture. After 3 days of first media exposure, glomerular outgrowth cells are cultured in a second media for 7 additional days with fresh second media changes occurring every 48 hours thereafter. Following the 10-day differentiation protocol, engineered podocytes-like cells are prevalent and impart filtration function in a bioengineered kidney, as shown in FIG. 1. TABLE 1 shows the components of the first media and the second media.

TABLE 1
First and Second Media Components

| First Media | Second Media |
|---|---|
| Dulbecco's Modified Eagle | Dulbecco's Modified Eagle |
| Medium/Nutrient | Medium/Nutrient |
| Mixture F-12 as media base | Mixture F-12 as media base |
| 1X insulin-Transferrin-Selenium | 1X insulin-Transferrin-Selenium |
| 1% Pen-Strep | 1% Pen-Strep |
| 10% Fetal Bovine Serum | 4 μM SB431542 |
| 1 μM Retinoic Acid | 2 μM IWR-1 |
| 100 nM Calcitriol | |
| 100 nM Dexamethasone | |

### Example 2: Engineered Podocyte-Like Cells are Functional in Engineered Kidneys

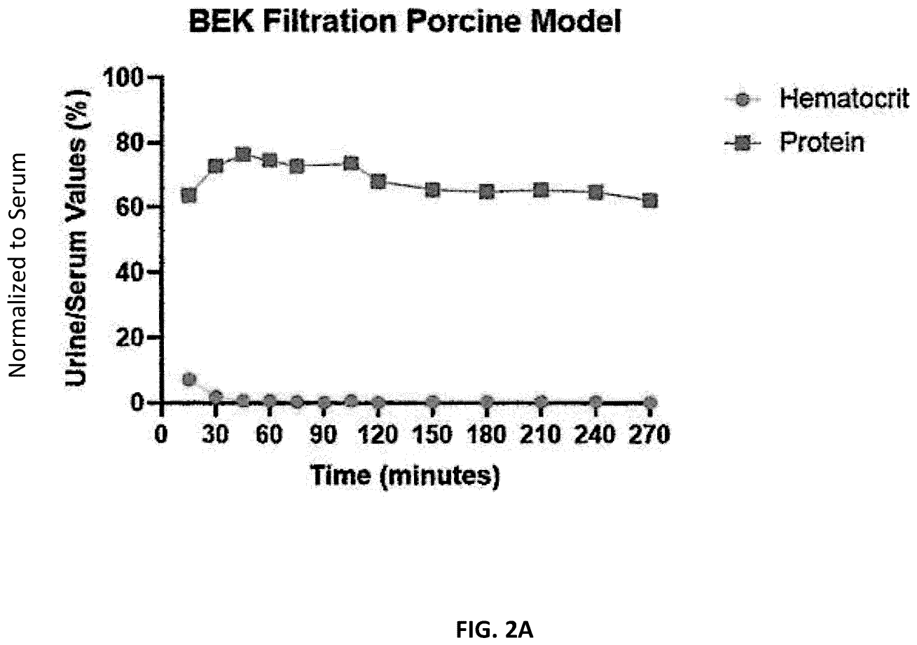

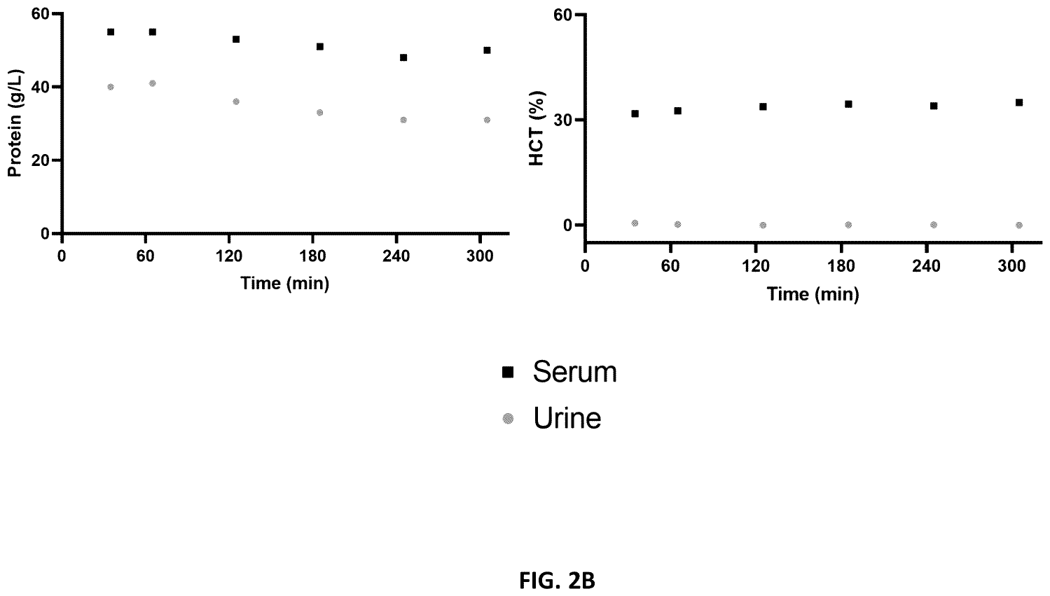

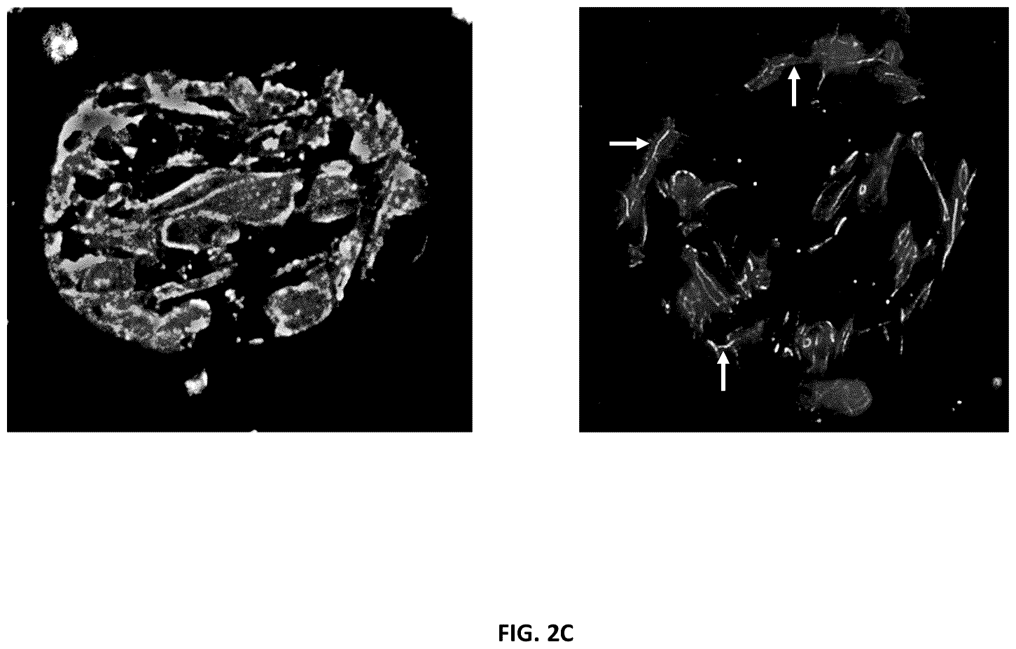

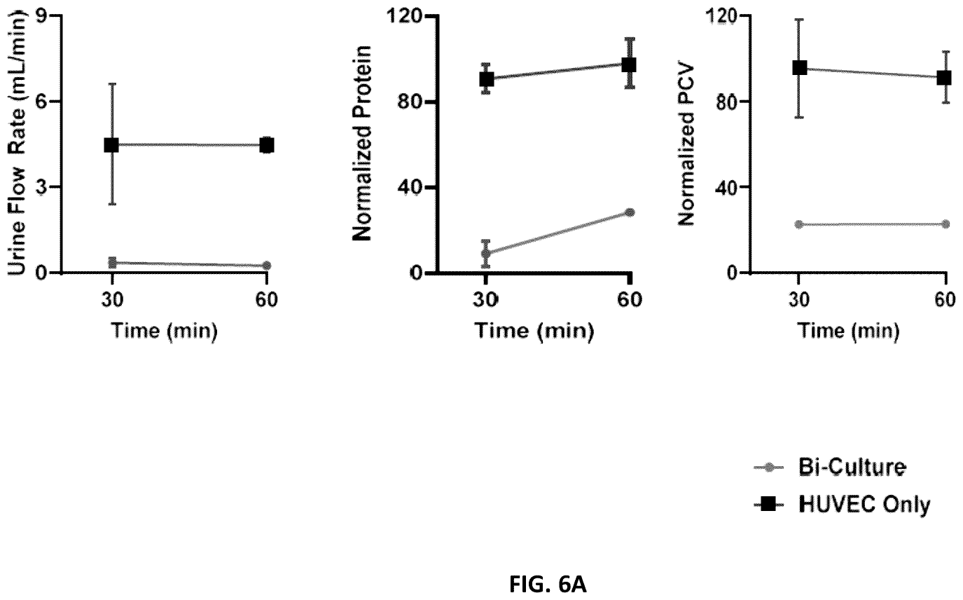

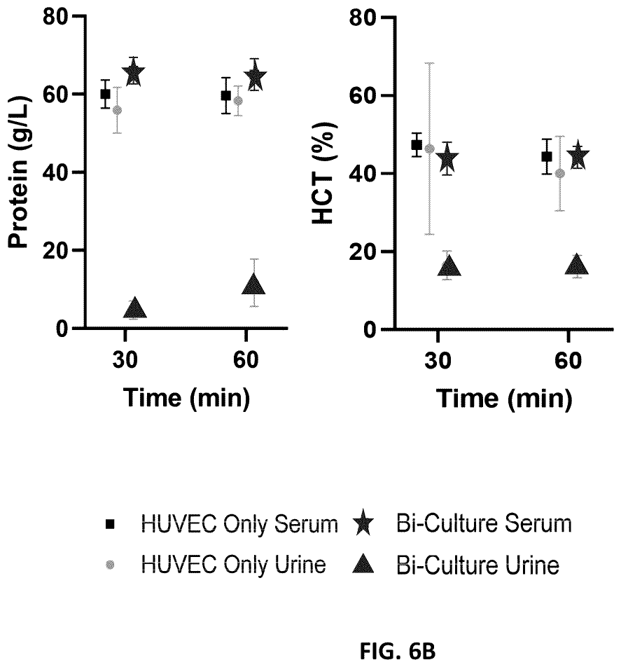

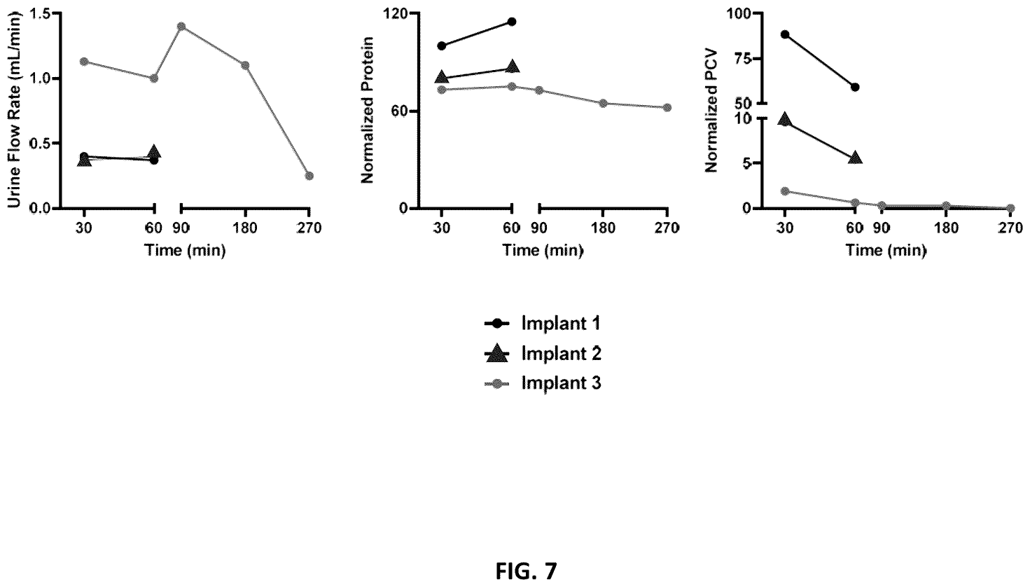

Normothermic and porcine perfusion were performed with bioengineered kidneys comprising engineered podocyte-like cells. The kidneys showed sustained filtration function e.g., greater than 4 hours of protein retention, which is shown by urine protein values being much lower than serum values, and sustained blood cell retention, which is shown by low (less than 15%) hematocrit in the urine as seen in FIG. 2B and FIG. 6B. The data in FIG. 2C shows bioengineered kidneys recellularized with engineered podocyte-like cells show repopulation of glomeruli with cells that express podocin, which is a protein used for podocyte function. Further the bioengineered kidneys are populated with cells that display primary processes as shown by the arrows. Primary processes are a unique and functionally relevant structural feature that podocytes possess, maintaining the complex cytoarchitecture that enables them to perform glomerular filtration.

### Example 3: Engineered Podocyte-Like Cells Show Podocyte Characteristics

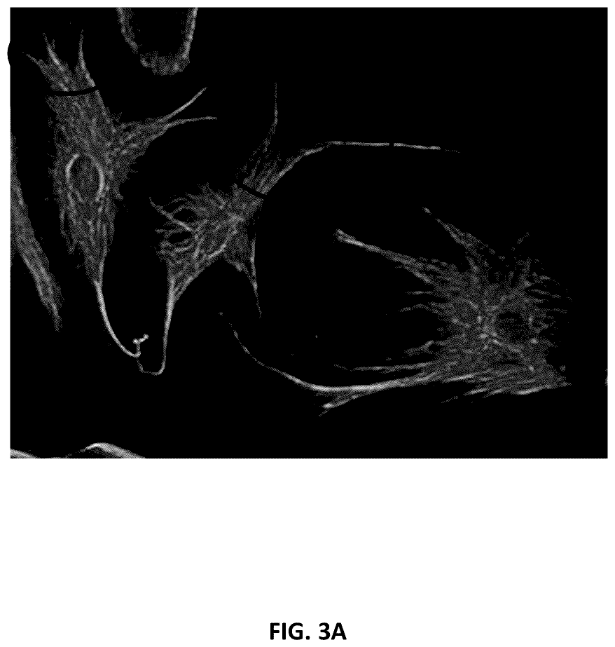

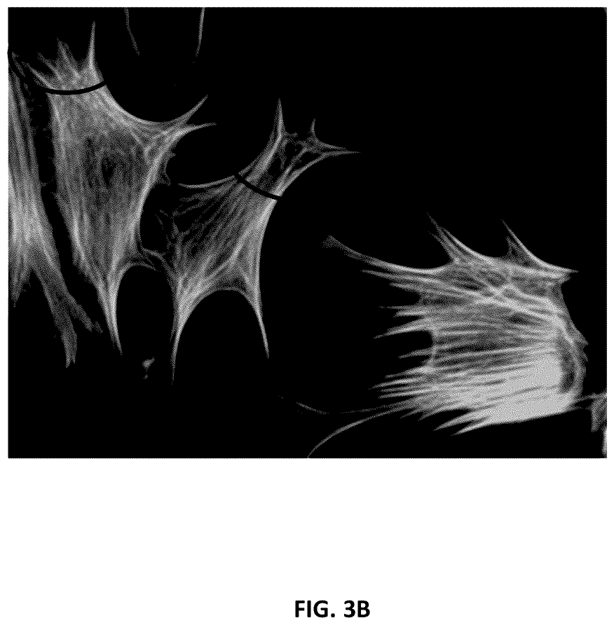

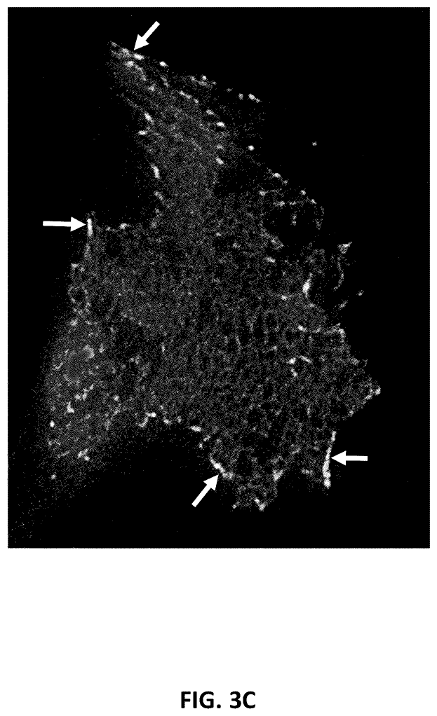

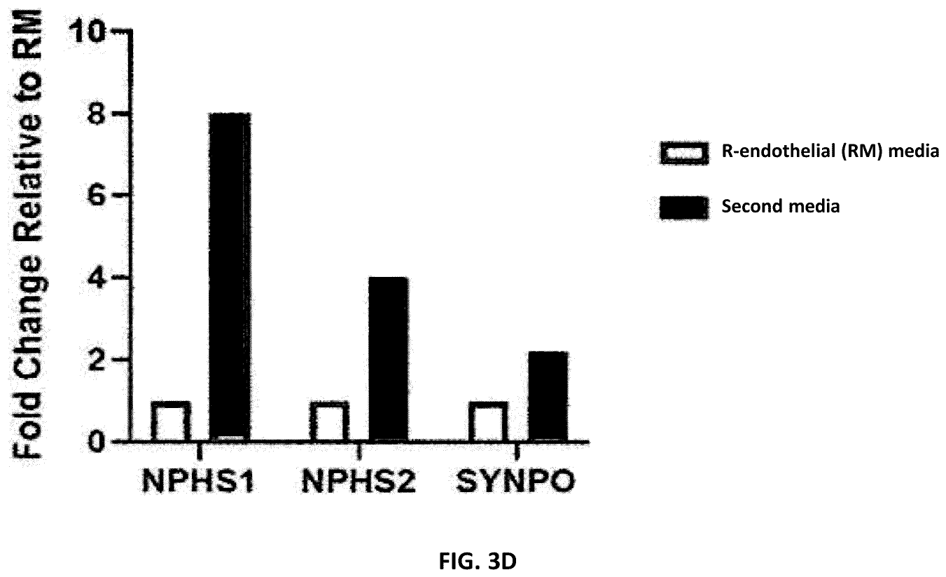

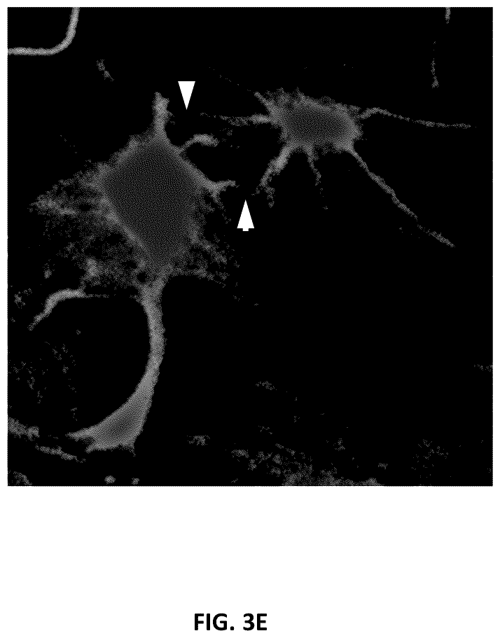

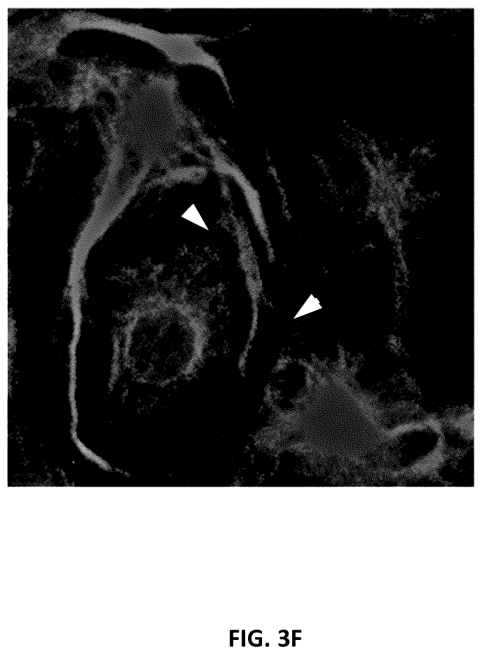

Engineered podocyte-like cells were differentiated as described in Example 1. The engineered podocyte-like cells were view by fluorescence microcopy to determine expression of podocyte proteins. FIG. 3A shows immunofluorescence labeling of second media-treated glomerular outgrowth cells (e.g., engineered podocyte-like cells) cultured on collagen I for F-actin and FIG. 3B shows immunofluorescence labeling for vimentin, which is a type III intermediate filament protein. These images show characteristic podocyte cytoskeletal organization with multiple extensions as shown in the circles. FIG. 3C shows second media-treated glomerular outgrowth cells (e.g., engineered podocyte-like cells) display prominent nephrin, a protein for filtration function. Nephrin expression was localized to the cell membrane as shown by the arrows. FIG. 3D shows second media-treated glomerular outgrowth cells (e.g., engineered podocyte-like cells) causes upregulation of several podocyte markers (NPHS1, NPHS2, and SYNPO) as show via quantitative reverse transcriptase Polymerase Chain Reaction (PCR). NPHS1 encodes for nephrin, NPHS2 encodes for podocin, and SYNPO encodes for synaptopodin. NPHS1 had about a 8 fold increase in expression after growth in second media compared to growth in RM media. NPHS2 had about a 4 fold increase in expression after growth in second media compared to growth in RM media. SYNPO had about a 2.5 fold increase in expression after growth in second media compared to growth in RM media. The second media-treated glomerular outgrowth cells were compared to glomerular outgrowth cells grown in standard R-endothelial media after a 6 day culture period. FIGS. 3E-3F show high magnification images of immunofluorescence labeling for vimentin that show areas (as indicated by arrowheads) where cell secondary processes, characteristic of podocytes, interact.

### Example 4: Differences Between Engineered Podocyte-Like Cells and Primary Podocytes

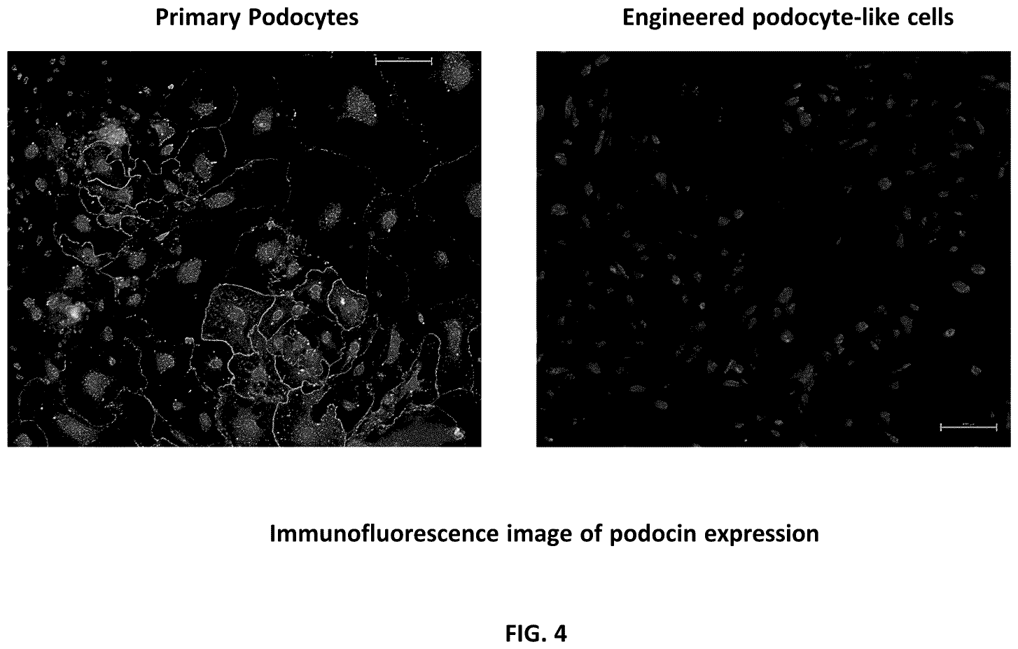

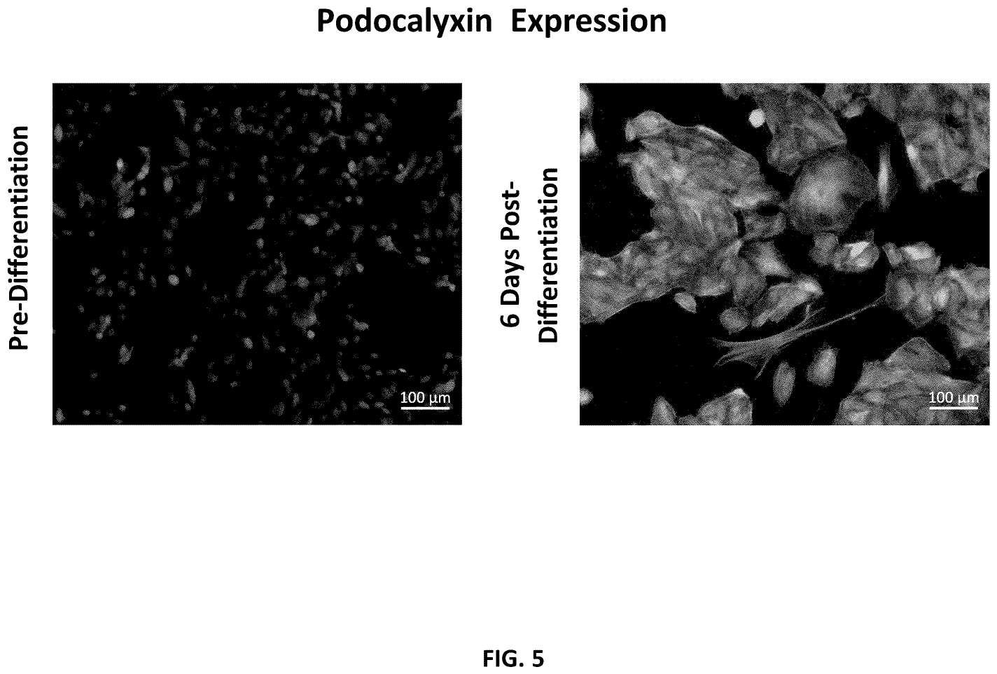

Fluorescence microcopy was used to determine the differences between engineered podocyte-like cells developed using the methods disclosed herein and primary podocytes obtained directly from a kidney. The expression of podocin was compared between the two cell types. As shown in FIG. 4, the primary podocytes (left image) had higher and more appropriately localized expression of podocin than the differentiated engineered podocyte-like cells (right image). Other podocyte proteins, such as nephrin and synaptopodin, show a similar trend (not shown). The images were taken from cells grown in culture. Thus, the images may suggest that the differentiated engineered podocyte-like cells may be missing biological factures, such as cellular factors and cellular interactions, present in native kidney cells that drive its unique phenotype.

### Example 5: Kidney Cell Seeding and Culture and Assessment of Filtration Function

To start kidney cell seeding, media was removed from the bioreactor chamber and replaced with fresh, prewarmed media, and perfusion was switched from the artery to the ureter.

Podocytes, such as engineered podocyte-like cells and mesangial cells were seeded into the decellularized, porcine kidney extracellular matrix through perfusion by the ureter. To allow for the seeded cells to reach the glomerulus of the decellularized, porcine kidney extracellular matrix, a slight vacuum (20-40 mmHg) was pulled on the bioreactor chamber while the kidney sat above the media volume. The perfusion was stopped, and the vacuum pulled a cell suspension from a sterile bottle through tubing hooked to the bioreactor chamber and into the ureter of the kidney. These cells were pulled up to the glomerulus, at which point they reached a physical barrier (i.e., glomerular basement membrane) keeping them within the glomerulus.

Recellularized kidneys were then continuously perfused with media. In some cases, if an operation is to be performed that requires pausing perfusion. The media in the bioreactor was replaced with fresh media every 24 hours once the first seeding began. During a media change, perfusion was paused, and the media was replaced with fresh, pre-warmed media. Similarly, when perfusion was changed to a different vessel conduit, the perfusion was paused while the connection was moved. A sample was collected every day prior to the media change to monitor glucose and the media volume was increased (up to the maximum volume allowed by the bioreactor assembly) as needed to ensure sufficient nutrients throughout the culture period

To assess filtration function of a bioengineered kidney an in-house closed loop normothermic perfusion system facilitated collection of both blood and urine samples to measure functionally relevant analytes, including creatinine, urea, sodium, potassium, total protein, hematocrit, glucose, lactate, and bicarbonate. Protein and hematocrit (HCT) readouts were specifically used to assess the degree of filtration performed by engineered podocyte-like cells.

### Example 6: Transplantation of a Bioengineered Kidney

A subject is diagnosed with kidney failure from a kidney disease. To treat the disease, the subject receives a transplant of a recellularized kidney comprising the engineered podocyte-like cells described herein. Once transplanted, the recellularized kidney has at least partial kidney function and the subject no longer has kidney failure.

### Example 7: Seeding Methods to Obtain Filtration Function

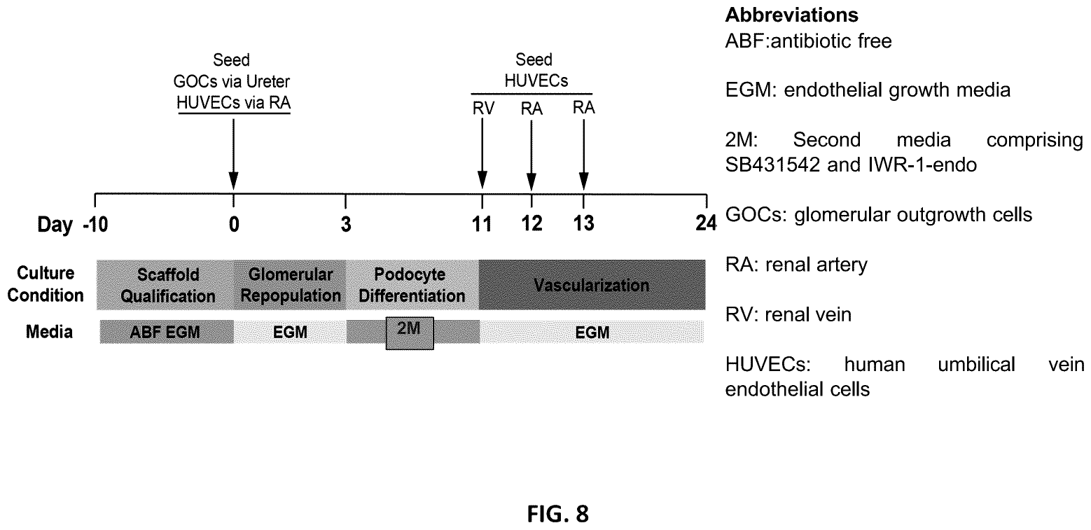

To prime the vascular passageways, decellularized porcine kidney grafts were perfused for 30 minutes on the renal artery with epithelial growth media (EGM) before HUVEC seeding. An initial seeding of 150 million HUVECs at a concentration of 2 million cells/mL was performed using manual syringe seeding at a rate of 50 mL/min. This seeding served to re-endothelialize the glomerular capillaries. The renal artery was perfused for another 30 minutes after HUVEC seeding. To prime the urinary passageways, grafts were perfused on the ureter with endothelial growth media (EGM) for 30 minutes before glomerular outgrowth cell (GOC) seeding. 500 million GOCs were seeded through the ureter for 30 minutes while the bioreactor was under −40 mmHg vacuum pressure. This seeding served to repopulate the urinary side of the glomerular basement membrane. Following the aforementioned seedings, grafts were cultured with EGM on days 0-2 of bioengineered kidney culture to proliferate HUVECs and GOCs. On Day 3 of bioengineered kidney culture, the media was changed from EGM to a second media (e.g., media comprising SB431542 and IWR-1-endo) to promote podocyte differentiation. Second media culture continued until day 8. On Day 9 of bioengineered kidney culture, the media was changed to a transitional media comprised of equal parts second media and EGM. A series of HUVEC seedings occurred on Days 10-12 of bioengineered kidney culture to re-endothelialize the renal vasculature: on Day 10, 150 million HUVECs were syringe seeded through the renal vein, on Day 11, 150 million HUVECs were syringe seeded through the renal artery, and on Day 12, 150 million HUVECs syringe seeded through the renal artery. Following the HUVEC seeding series, bioengineered kidneys were perfused through the artery with EGM at a flow rate starting at 50 mL/min and sequentially stepped up by 50 mL/min daily until either the flow rate or arterial pressure reached a maximum of 500 mL/min or 80 mmHg, respectively. Perfusion was maintained at these maximums for the remainder of the bioengineered kidney culture period (day 24-25). Throughout the bioengineered kidney culture period, the second media was refreshed every other day and EGM was refreshed with daily media changes. A summary of the development of a bioengineered kidney graft using the methods herein is shown in FIG. 8.

### Example 8: Functional Testing Via Normothermic Machine Perfusion

Porcine blood was circulated by a digitally controlled pump, maintained at a temperature of 37° C. by a water bath circulator (and ventilated with 20% O₂, 5% CO₂, & 75% N₂ gas mixture through an oxygenator. The gas mixture flow rate was regulated at 150 mL/min using a steel-ball rotameter. From a blood reservoir, the blood was propelled by the pump through the oxygenator into the renal artery and exited the kidney through the renal vein circulating back to the blood reservoir. A target arterial pressure of 100 mmHg was maintained by a CompactRIO-based PID system. The resulting perfusion flow rates were between 300 mL/min and 800 mL/min. Pressure and flow rate data were continuously captured via custom software. Every 15 minutes for 1 hour, urine and perfusate samples were collected. Samples were processed to obtain plasma and stored at −80° C. for subsequent analysis.

### Example 9: Implant Methods

The peritoneum of the porcine animal was opened with a midline incision, and the kidney was implanted heterotopically at the inferior vena cava (IVC) and abdominal aorta (AA). The ureter was routed to a 0.25 inch diameter silicone tubing that was connected to a 50 ml conical tube for effluent collection. Following graft reperfusion, samples were taken of the perfusing porcine blood and collected urine to assess kidney function.

### Example 10: Additional Exemplary Podocyte Differentiation Protocol

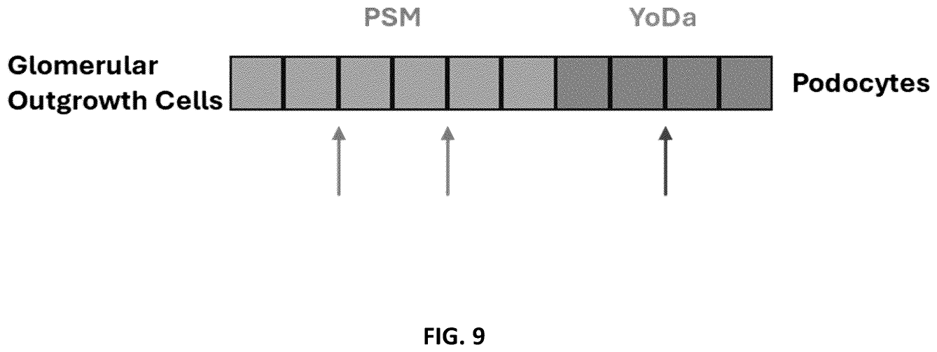

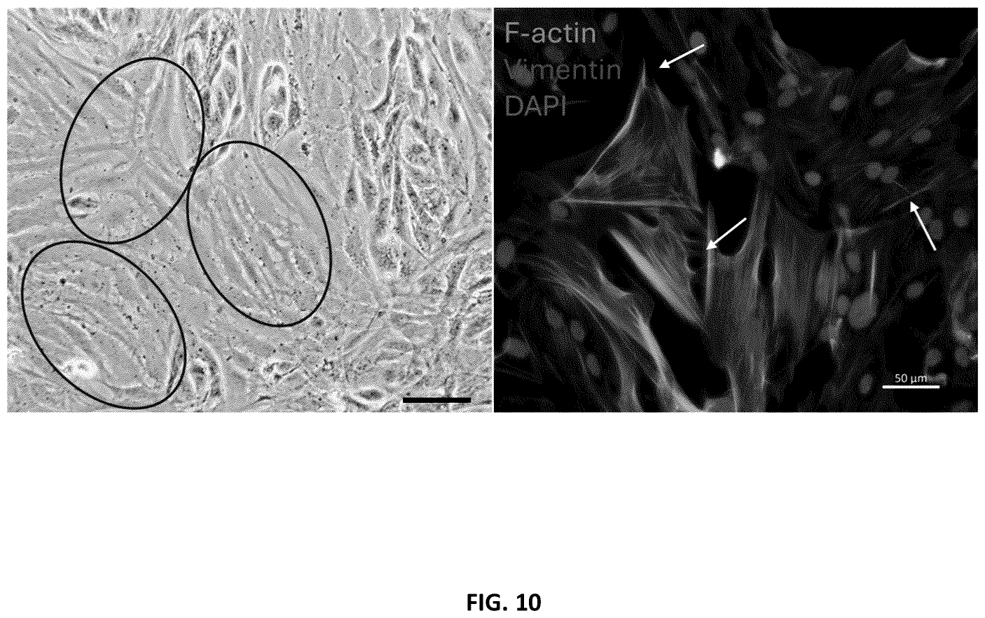

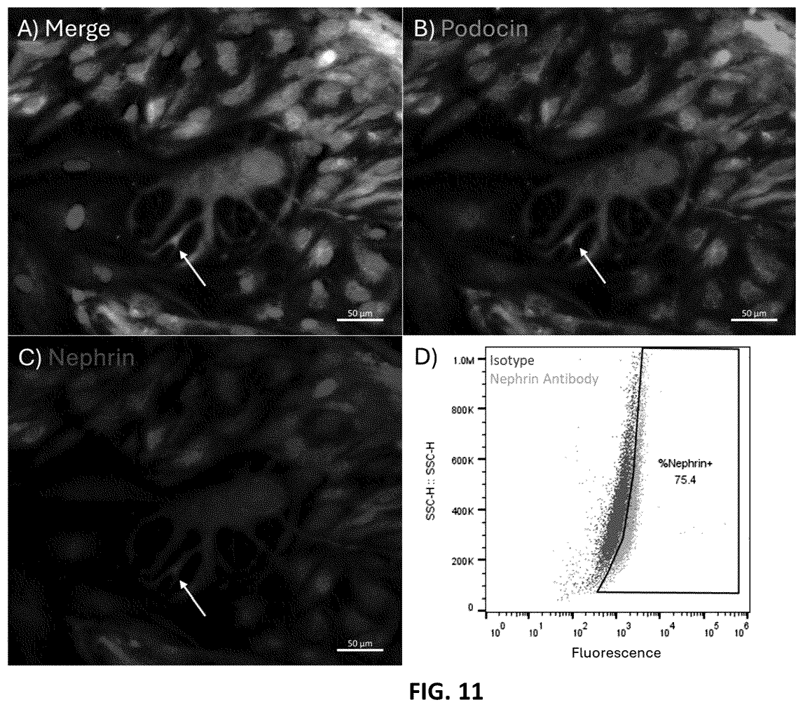

To initiate the podocyte differentiation protocol, glomerular outgrowth cells were cultured in Podocyte Specification Media (PSM) for 4 to 6 days with a media change with PSM occurring every 48 hours after initial culture. After 4 to 6 days of PSM media exposure, glomerular outgrowth cells were cultured in YoDa media for 2 to 4 additional days, changing YoDa media every 48 hours. FIG. 9 depicts the media scheme used to induce transdifferentiation of glomerular outgrowth cells into podocytes, with each square representing one day of culture and arrows indicating when media changes should occur with a particular media. After the 6-10 day culture period, podocytes were prevalent, as shown in FIGS. 10 and 11. Table 2 shows components and exemplary amount/concentrations for the components of the PSM and YoDa media used in this example.

TABLE 2
PSM and YoDa Media Components

| Podocyte Specification Media | |
|---|---|
| (PSM) | YoDa Media |
| Dulbecco's Modified Eagle | Dulbecco's Modified Eagle |
| Medium/Nutrient | Medium/Nutrient |
| Mixture F-12 as media base | Mixture F-12 as media base |
| 1X Insulin-Transferrin-Selenium | 1X Insulin-Transferrin- Selenium |
| 1% Pen-Strep | 1% Pen-Strep |
| 4 μM SB431542 | 15 to 40 U/mL Heparin |
| 2 μM IWR-1 | 0.2 μM Retinoic Acid |
| | 10 μM Y-27632 (ROCK inhibitor) |

### Example 11: Additional Exemplary Podocyte Differentiation Protocol

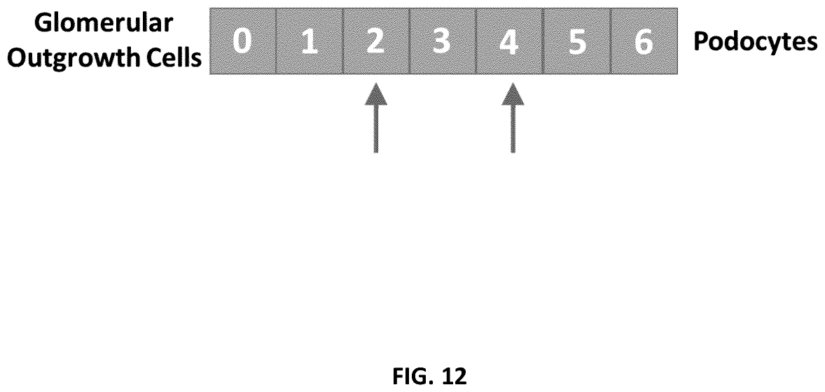

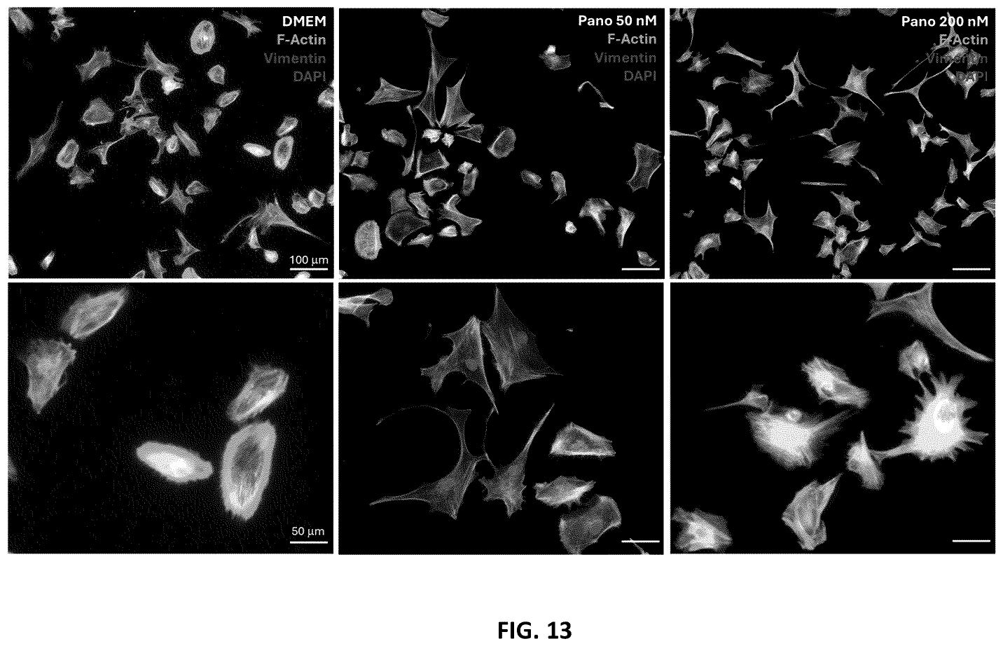

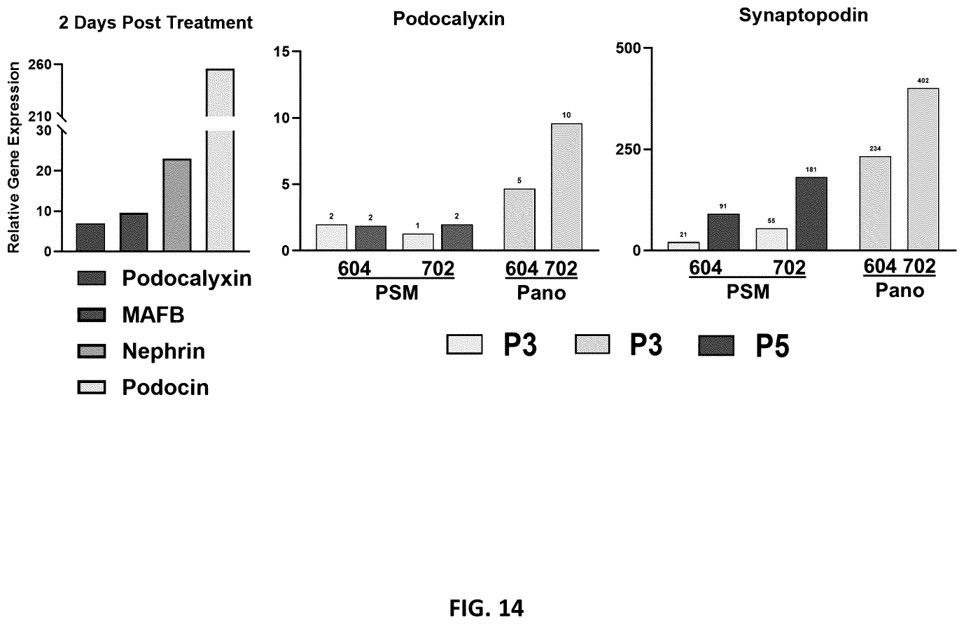

To initiate the podocyte differentiation protocol, culture glomerular outgrowth cells in Panobinostat media for 2 to 6 days with a media change with Panobinostat media occurring every 48 hours after initial culture. FIG. 12 depicts the media scheme to induce transdifferentiation of glomerular outgrowth cells into podocytes with each square representing one day of culture and arrows indicating when media changes should occur with a particular media. After the culture period, podocytes were present, as shown in FIGS. 13 and 14. Table 3 shows the components of the Panobinostat media.

TABLE 3
Panobinostat Media Components
Panobinostat Media

Dulbecco's Modified Eagle Medium/Nutrient
Mixture F-12 as media base
1X Insulin-Transferrin- Selenium
1% Pen-Strep
50 nM to 200 nM Panobinostat

### Example 12: Podocyte Maintenance

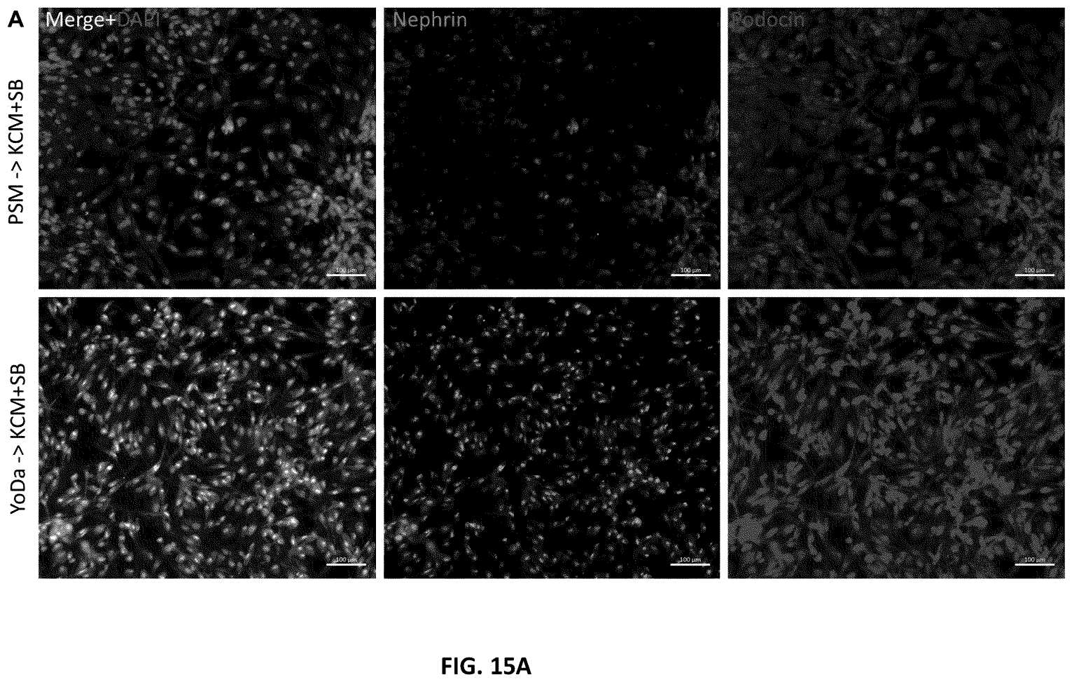

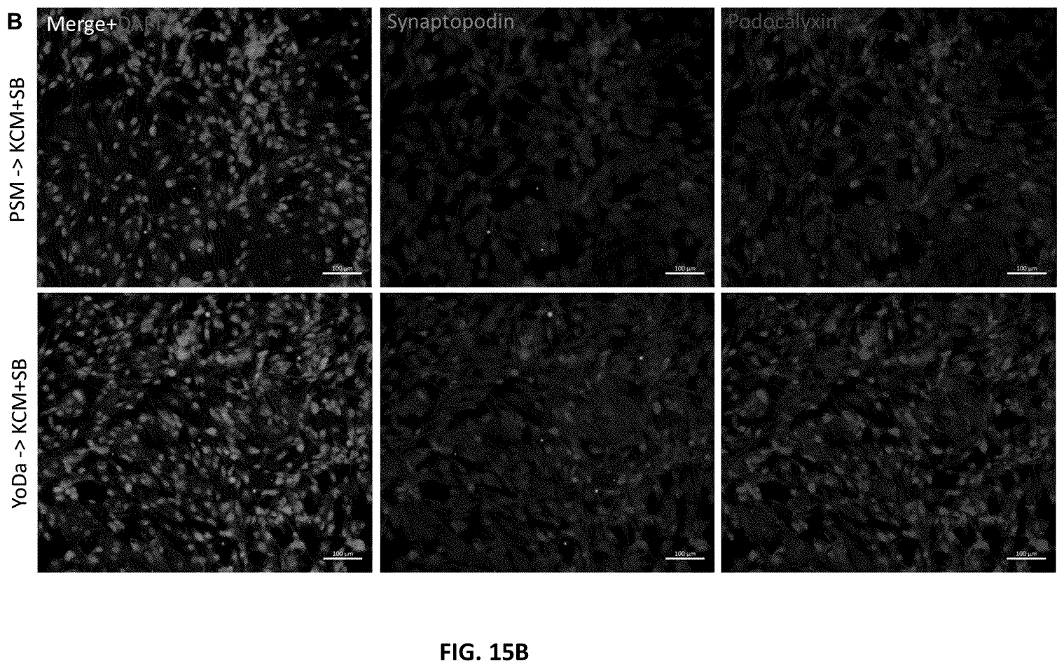

The following podocyte maintenance media provides a superior and innovative solution to maintaining the phenotype of differentiated podocytes in co-culture with other kidney cells for our kidney grafts. The protocol below specifies how cell culture media is formulated and used to maintain differentiated podocytes. After completing podocyte differentiation, podocytes were cultured in Kidney Co-culture Media (KCM)+4 μM SB431542 for up to 14 days with a media change occurring every other day. See FIG. 15. Table 4 shows the components of the maintenance media.

TABLE 4
Maintenance Media Components.

| | Kidney Co-culture Media (KCM) + SB |
|---|---|
| KCM | R&D Systems Endothelial Base Media |
| | 1% Penicillin-Streptomycin |
| | 2% Fetal Bovine Serum |
| | 1.05 U/mL Heparin |
| | 50 μg/mL Ascorbic Acid |
| | 1.15 μg/mL Hydrocortisone |
| | 20 ng/mL rh FGF (e.g., E. coli-derived human |
| | FGF basic, Pro143-Ser288, with an N- |
| | terminal Ala, Accession# P09038) |
| | 5 ng/mL rh VEGF (e.g., Sf21 (baculorvirus)- |
| | derived human VEGF 165, Ala27-Arg191, |
| | Accession # NP_001165097) |
| | 15 ng/mL rh EGF (e.g., E.coli-derived human |
| | EGF protein, Asn971-Arg1023, with an N- |
| | terminal Met, Accession # P01133) |
| | 15 ng/ml Long R3 IGF (e.g., E.coli-derived |
| | human IGF-I, Gly49-Ala118 (Glu51Arg), N- |
| | terminus MFPAMPLSSLFVN, Accession # |
| | P05019.1) |
| | 0.125 U/mL Insulin (from bovine pancreas) |
| | 10 nM Triiodothyronine (T3) |
| | 1 μM Epinephrine |
| | 5 μg/mL Holo-transferrin |
| +SB | 4 μM SB431542 |

Amino Acid Sequences
SEQ ID NO: 1
PALPEDGGSGAFPPGHFKDPKRLYCKNGGFFLRIHPDGRVDGVREKSDP
HIKLQLQAEERGVVSIKGVCANRYLAMKEDGRLLASKCVTDECFFFERL
ESNNYNTYRSRKYTSWYVALKRTGQYKLGSKTGPGQKAILFLPMSAKS
SEQ ID NO: 2
APMAEGGGQNHHEVVKFMDVYQRSYCHPIETLVDIFQEYPDEIEYIFKP
SCVPLMRCGGCCNDEGLECVPTEESNITMQIMRIKPHQGQHIGEMSFLQ
HNKCECRPKKDRARQENPCGPCSERRKHLFVQDPQTCKCSCKNTDSRCK
ARQLELNERTCRCDKPRR
SEQ ID NO: 3
NSDSECPLSHDGYCLHDGVCMYIEALDKYACNCVVGYIGERCQYRDLKW
WELR
SEQ ID NO: 4
GPETLCGAELVDALQFVCGDRGFYFNKPTGYGSSSRRAPQTGIVDECCF
RSCDLRRLEMYCAPLKPAKSA

While preferred embodiments of the present disclosure have been shown and described herein, such embodiments are provided by way of example only. Numerous variations, changes, and substitutions will occur without departing from the disclosure. Various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of making engineered podocyte-like cells, the method comprising:
a) culturing glomerular cells in a first media comprising a retinoic acid, a corticosteroid, a calcitriol, or a salt of any one of these for about 2 to about 4 days;
b) removing the glomerular cells from the first media; and
c) culturing the glomerular cells in a second media comprising a SB431542, a salt thereof, an IWR-1-endo, or a salt thereof for about 6 to about 12 days,
wherein the culturing of the glomerular cells in the second media for about 6 to about 12 days results in differentiation of the glomerular cells into the engineered podocyte-like cells
that have increased expression of one or more of podocin, nephrin, podocalyxin, or synaptopodin, as compared to the glomerular cells prior to culturing in the first media.

2. The method of claim 1, wherein the first media comprises a dexamethasone or a salt thereof.

3. (canceled)

4. (canceled)

5. (canceled)

6. (canceled)

7. (canceled)

8. (canceled)

9. (canceled)

10. (canceled)

11. (canceled)

12. The method of claim 1, wherein the engineered podocyte-like cells have increased gene expression of NPHS1, NPHS2, SYNPO, or any combination thereof, as compared to the glomerular cells.

13. (canceled)

14. (canceled)

15. The method of claim 1, wherein the engineered podocyte-like cells comprise a cytoskeletal organization with multiple extensions as compared to a cytoskeletal organization of glomerular cells.

16. (canceled)

17. (canceled)

18. The method of claim 1, wherein the engineered podocyte-like cells have decreased expression of one or more of podocin, nephrin, podocalyxin, or synaptopodin as compared to primary podocytes.

19. (canceled)

20. (canceled)

21. (canceled)

22. (canceled)

23. (canceled)

24. (canceled)

25. (canceled)

26. (canceled)

27. (canceled)

28. (canceled)

29. The method of claim 1, wherein the glomerular cells are engrafted onto an least partially decellularized kidney extracellular matrix prior to the culturing glomerular cells in a first media.

30. (canceled)

31. An engineered podocyte-like cell, wherein the engineered podocyte-like cell comprises:
a) increased expression of one or more of podocin, nephrin, podocalyxin or synaptopodin as compared to a glomerular cell; and
b) decreased expression of one or more of podocin, nephrin, podocalyxin or synaptopodin, as compared to a primary podocyte cell.

32. The engineered podocyte-like cell of claim 31, wherein the engineered podocyte-like cells comprise a cytoskeletal organization with multiple extensions as compared to the cytoskeletal organization of the glomerular cell.

33. The engineered podocyte-like cell of claim 31, wherein the engineered podocyte-like cells have increased gene expression of NPHS1, NPHS2, SYNPO, or any combination thereof as compared to the glomerular cell.

34. The engineered podocyte-like cell of claim 31, wherein the engineered podocyte-like cells have decreased localization of one or more of podocin, nephrin, podocalyxin, or synaptopodin as compared to the primary podocytes.

35. An engineered podocyte-like cell made by the method of claim 1.

36. A method of engrafting cells on an at least partially decellularized kidney extracellular matrix, the method comprising:
contacting the at least partially decellularized kidney extracellular matrix with a plurality of the engineered podocyte-like cells of claim 31.

37. The method of claim 36, wherein the contacting occurs in a bioreactor chamber, and wherein the contacting comprises depositing the plurality of the engineered podocyte-like cells suspended in an aqueous composition through a ureter and into a glomerulus of the at least partially decellularized kidney extracellular matrix, thereby engrafting cells on the at least partially decellularized kidney extracellular matrix.

38. The method of claim 36, wherein the method further comprises seeding a plurality of mesangial cells, a plurality of human umbilical vein endothelial cells (HUVEC), or both.

39. The method of claim 37, wherein the depositing through the ureter is performed under reduced pressure in the bioreactor chamber.

40. The method of claim 37, wherein the method further comprises continuously perfusing a media through the at least partially decellularized kidney extracellular matrix after the engrafting.

41. (canceled)

42. An at least partially recellularized isolated organ or portion thereof comprising the engineered podocyte-like cell of claim 31, wherein the at least partially recellularized isolated organ or portion thereof in a closed loop normothermic perfusion system
a) sustains urine/serum protein values in urine of less than 30% at 1 hour post normothermic perfusion, and less than 65% at 4 hours post implantation; or
b) sustains urine/serum hematocrit levels in urine of less than 30% at 1 hour post normothermic perfusion, and less than 1% at 4 hours post implantation.

43. (canceled)

44. (canceled)

45. (canceled)

46. (canceled)

47. (canceled)

48. (canceled)

49. (canceled)

50. A method of making engineered podocyte-like cells, the method comprising:
a) culturing glomerular cells in a first media comprising a transforming growth factor beta pathway inhibitor, and a Wnt pathway inhibitor, or a salt of either of these for about 4 to about 8 days; and
b) removing the glomerular cells from the first media; and
c) culturing the glomerular cells in a second media comprising a retinoic acid, a Rho kinase (ROCK) inhibitor, or a salt of either of these for about 2 to about 6 days, wherein the culturing of the glomerular cells in the second media for about 2 to about 6 days results in differentiation of the glomerular cells into the engineered podocyte-like cells.

51. The method of claim 50, wherein the engineered podocyte-like cells have increased interdigitating foot processes as compared to the glomerular cells prior to culturing in the first media.

52. The method of claim 50, wherein the engineered podocyte-like cells express at least one of F-actin and vimentin.

53. The method of claim 50, wherein the engineered podocyte-like cells express at least one of podocin, nephrin, podocalyxin, or synaptopodin.

54. The method of claim 50, wherein the transforming growth factor beta pathway inhibitor comprises SB431542.

55. (canceled)

56. (canceled)

57. The method of claim 50, wherein the Wnt pathway inhibitor comprises IWR-1.

58. (canceled)

59. (canceled)

60. (canceled)

61. (canceled)

62. The method of claim 50, wherein the ROCK inhibitor comprises Y-27632.

63. The method of claim 50, wherein the glomerular cells are engrafted onto an least partially decellularized kidney extracellular matrix prior to the culturing glomerular cells in a first media.

64. An engineered podocyte-like cell made by the method of claim 50.

65. (canceled)

66. (canceled)

67. (canceled)

68. (canceled)

69. A method of making engineered podocyte-like cells, the method comprising:
culturing glomerular cells in a media comprising a histone deacetylase inhibitor for at least about 4 to about 8 days, wherein the culturing results in differentiation of the glomerular cells into the engineered podocyte-like cells.

70. The method of claim 69, wherein the histone deacetylase inhibitor comprises a hydroxamic acid or a salt thereof.

71. The method of claim 69, wherein the histone deacetylase inhibitor comprises Panobinostat or a salt thereof.

72. The method of claim 69, wherein the glomerular cells are engrafted onto an least partially decellularized kidney extracellular matrix prior to the culturing glomerular cells.

73. An engineered podocyte-like cell made by the method of claim 69.

74. (canceled)

75. (canceled)

76. (canceled)

77. (canceled)

78. A method of maintaining engineered podocyte-like cells, the method comprising culturing the engineered podocyte-like cells in media comprising ascorbic acid, hydrocortisone, rh FGF, rh VEGF, rh EGF, Long R3 IGF, insulin, triiodothyronine, epinephrine, holo-transferrin, and SB431542.

79-119. (canceled)
